(19) United States
(12) Patent Application Publication
(10) Pub. No.: US 2025/0109905 A1
(43) Pub. Date: Apr. 3, 2025

(54) LYOPHILIZATION USING CONTROLLED NUCLEATION

(71) Applicant: Regeneron Pharmaceuticals, Inc., Tarrytown, NY (US)

(72) Inventors: Xiaolin Tang, Old Tappan, NJ (US); Yao Chen, Whitestone, NY (US); Mary Kleppe, Milford, CT (US); Sarah Ingram, New York, NY (US); Saurav Sharma, Danbury, CT (US)

(21) Appl. No.: 18/898,912

(22) Filed: Sep. 27, 2024

Related U.S. Application Data
(60) Provisional application No. 63/681,328, filed on Aug. 9, 2024.
(60) Provisional application No. 63/541,573, filed on Sep. 29, 2023.

(51) Int. Cl. F26B 5/06; A61K 9/19

(52) U.S. Cl. CPC F26B 5/06; A61K 9/19

(57) ABSTRACT

The present inventions relate to improved lyophilization methods for therapeutic proteins, such as monoclonal antibodies, using controlled nucleation. The controlled nucleation approach is time efficient, and produces lyophilized products with high quality and faster reconstitution times. The present inventions also relate to efficient lyophilization process to achieve moderate moisture content in room temperature stable therapeutic proteins.

This application claims the benefit of priority to U.S. Application Ser. No. 63/541,573, filed Sep. 29, 2023; and U.S. Application Ser. No. 63/681,328, filed Aug. 9, 2024. These applications are incorporated by reference in their entirety.

### FIELD OF THE INVENTIONS

The present inventions relate to improved lyophilization methods for therapeutic proteins using controlled nucleation.

### BACKGROUND OF THE INVENTIONS

Therapeutic proteins include Fc-containing proteins, such as monoclonal antibodies (mAb) and receptor Fc-fusion proteins, such as trap proteins, are used as therapeutics proteins. Lyophilization is an accepted approach for long-term storage of therapeutic proteins. Present platform lyophilization approaches (that is, current standard lyophilization) are time consuming, and often can result in lyophilized preparations that have quality issues. The present inventions provide improved lyophilization methods and improved lyophilized therapeutic products that do not suffer from the drawbacks of conventional platform technologies. The present inventions also provide room temperature stable lyophilized therapeutic protein products, methods of making the same, and methods of obtaining optimal moisture content in the therapeutic products in order to the make room temperature stable lyophilized therapeutic products.

### SUMMARY OF THE INVENTIONS

The inventions provide methods of lyophilizing a protein product, wherein the method comprises the steps: (a) cooling a solution comprising a protein product below the freezing point of the solution; (b) pressuring the cooled solution of step (a) with a gas; (c) releasing the pressure of step (b) to allow nucleation, thereby resulting ice nuclei to form in the cooled solution; and (d) lyophilizing the cooled solution of step (c) to form a lyophilized protein product. The gas can be selected from the group consisting of air, helium, nitrogen or argon. During step (b) the gas can be present at 14 to 42 psig, preferably 25 to 30 psig and more preferably about 28 psig.

The cooled solution can be in a lyophilization vial. Step (c) can be conducted at a temperature of −2° C. to −10° C., preferably at about −5° C. The temperature can be maintained for about an hour or less, such as 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 minutes or less. If desired, the temperature can be maintained for more than an hour.

The protein can be an Fc-containing protein, such as an Fc-fusion protein or an antibody. The antibody is preferably a monoclonal antibody.

Where the Fc-containing protein is a monoclonal antibody, the antibody can be reconstituted to 150 mg/ml in about 3 minutes, in about 2 minutes and 50 seconds, in about 2 minutes and 40 seconds, in about 2 minutes and 30 seconds, in about 2 minutes and 20 seconds, in about 2 minutes and 10 seconds, in about 2 minutes, in about 1 minute and 50 seconds, in about 1 minute and 45 seconds, in about 1 minute and 40 seconds or less, as well as additional subranges made based upon the above values.

Lyophilized proteins produced by the methods also are provided. Preferably, the proteins can be Fc-containing proteins, such as antibodies and Fc-fusion proteins, for example. The inventions are further described and discussed below.

The inventions also provide methods of making a room temperature stable lyophilized therapeutic protein product by providing a target residual moisture content in the room temperature stable lyophilized therapeutic protein product, wherein the method comprises the steps of: (a) controlling the shelf temperature (Ts) after ice sublimation without a secondary drying step; and (b) controlling the duration of post primary drying time; wherein the target residual moisture content in the lyophilized therapeutic protein product is about 1% to about 7%. The target residual moisture content in the lyophilized therapeutic protein product can be about 2% to 6%, about 2% to 5%, about 2% to 4%, about 2% to 3%, about 3% to 7%, about 3% to 6%, about 3% to 5%, about 3% to 4%, about 4% to 7%, about 4% to 6%, about 4% to 5%, about 5% to 7%, about 5% to 6%, about 6% to 7%, about 1.0%, about 1.5%, about 2.0%, 2.5%, about 3.0%, about 3.5%, about 4.0%, about 4.5%, about 5.0%, about 5.5%, about 6.0%, about 6.5%, or about 7.0%, as well as additional subranges made based upon the above values. The stability of the lyophilized therapeutic protein product can be measured by determining change in % HMW protein aggregates in the product. The change in % HMW protein aggregates in the stable lyophilized therapeutic protein product is less than 1.0% as determined by Size Exclusion Chromatography-Ultra Performance Liquid Chromatography (SEC-UPLC). The change in % HMW protein aggregates in the stable lyophilized therapeutic protein product is about 1.0%, about 0.9%, about 0.8%, about 0.7%, about 0.6%, about 0.5%, about 0.4%, about 0.3%, about 0.2%, about 0.1% or about 0%, as well as additional subranges made based upon the above values. The inventions also provide room temperature stable lyophilized therapeutic protein product produced by any of the methods disclosed herein.

### BRIEF DESCRIPTION OF THE FIGURES

FIG. 1A is a graph depicting lyophilization process with annealing step included. FIGS. 1B and 1C provide a comparison between conventional and controlled nucleation methodologies on multiple lots of a monoclonal antibody preparation. FIG. 1B depicts data from a conventional freezing platform. FIG. 1C depicts data from controlled nucleation.

FIGS. 2A and 2B depict data from controlled nucleation parameters profiles on multiple lots of a monoclonal antibody preparation. FIG. 2A is at −5° C. and FIG. 2B is at −10° C.

FIGS. 3A to 3D depict data from various lyophilization approaches on multiple lots of a monoclonal antibody preparation. FIG. 3A depicts data from a conventional platform lyophilization. FIG. 3B depicts data from controlled nucleation at −5° C. FIG. 3C depicts data from a controlled nucleation at −10° C. FIG. 3D depicts data from a conventional platform lyophilization with annealing (see encircled between 0.0 and 20.0 hours). Arrows in each figure indicate the end of primary drying.

FIG. 4 depicts primary drying times and freezing plus primary drying times for conventional platform (see FIG. 3A), controlled nucleation at −5° C. (see FIG. 3B), controlled nucleation at −10° C. (see FIG. 3C), and conventional platform with annealing (see FIG. 3D).

FIGS. 5A and 5B depict size exclusion chromatography (SEC) data for percent native mAb using buffers comprising 150 mg/ml mAb and 5% sucrose or 1.5% sucrose. FIG. 5A shows results from samples stored at 25° C. and FIG. 5B shows results from samples stored at 40° C. FIGS. 5C and 5D depict size exclusion chromatography (SEC) data for Formulations F1, F2, F3, and F4 (See formulations in Table 1). FIG. 5C is at 25° C. and FIG. 5D is at 40° C.

FIGS. 6A and 6B: FIG. 6A shows platform lyophilized mAbs preparations in vials: Platform Drug Product (DP), Low concentration Placebo Product (PP), Low concentration Drug Product and Platform Placebo Product from left to right. See formulations in Table 1. The arrows in the figures indicate product shrinkage. FIG. 6B depicts lyophilized mAbs preparations in vials: Placebo Drug Product and Low concentration Drug Product (DP) using lyophilization with annealing at −5° C., with no annealing controlled nucleation, and controlled nucleation at −5° C. The arrows in the figures indicate product shrinkage.

FIG. 7 shows lyophilized mAbs preparations in vials prepared using controlled nucleation: Platform Drug Product, Low concentration Placebo Product, Platform Placebo Product and Low concentration Drug Product from left to right. See formulations in Table 1. The top row was performed at −5° C. and the bottom row was performed at −10° C.

FIG. 8 shows platform lyophilized mAbs preparations in vials with annealing performed prior to lyophilization: Low concentration Drug Product (DP), Platform Drug Product, Low concentration Placebo Product (PP), and Platform Placebo Product from left to right. See formulations in Table 1. The arrow in the figure indicates product on wall.

FIG. 9 shows platform lyophilized mAb preparations and lyophilized mAb preparations in vials made with controlled nucleation at −5° C. using the 5% w/v sucrose formulation (left) and the 1.5% sucrose formulation (right). The arrow in the figure indicates product on wall for conventional lyophilization.

FIGS. 10A and 10B show scanning electron micrographs (SEM) at the same scale. FIG. 10A is from a platform lyophilized (conventional) preparation and FIG. 10B is from a lyophilized preparation made with controlled nucleation at −5° C. See formulations in Table 1.

FIGS. 11A and 11B show scanning electron micrographs (SEM) at the same scale. FIG. 11A is from a lyophilized preparation made with controlled nucleation at −5° C., and FIG. 11B is from a lyophilized preparation made with controlled nucleation at −10° C. See formulations in Table 1.

FIGS. 12A and 12B show scanning electron micrographs (SEM) at the same scale. FIG. 12A is from a conventional platform lyophilization. FIG. 12B is from a conventional platform lyophilization with annealing at −5° C.

FIG. 13 is a graph depicting data on the effect of annealing on dry product resistance for 5% Glycine.

FIGS. 14A and 14B are figures depicting data concerning moisture content and stabilization of drug formulation at 5° C., 25° C. and 30° C. (FIG. 14A) and at 37° C. and 50° C. (FIG. 14B).

FIGS. 15A and 15B depict efficient lyophilization process to achieve moderate moisture content. End of primary drying when vapor composition is mainly nitrogen. Pirani Gauge Pressure≈Chamber Pressure measured by capacitance. FIG. 15A indicates STOP following the end of ice sublimation.

FIG. 16 is a graph depicting optimal combination of shelf temperature and post ice sublimation time.

FIG. 17 is a graph depicting % HMW, as determined by SE-UPLC, in stable lyophilized DP stored at 25° C. with moderate moisture content.

FIG. 18 are bar graphs depicting data from micro-flow imaging (MFI) analytical assays on ≥10 μm unfiltered particles and the impact on subvisible particles.

FIG. 19 are bar graphs depicting data from micro-flow imaging (MFI) analytical assays on ≥25 μm unfiltered particles and the impact on subvisible particles.

FIGS. 20A and 20B are graphs showing % HMW aggregates in platform DP (FIG. 20A) and low concentration DP (FIG. 20B), stored at a temperature of 25° C. under a Relative Humidity (RH) of 60% for over six months, as determined by SEC.

FIGS. 21A and 21B are graphs showing % Main (Region 2) in platform DP (FIG. 21A) and low concentration DP (FIG. 21B) over six months of storage as determined by cation exchange chromatography (CEX).

FIGS. 22A and 22B are graphs showing impact of 5% cryoprotectant on 25 μm (FIG. 22A) and 10 μm (FIG. 22B) particles in DP formulations, at different temperatures (25° C. and 40° C.) and times (at to, 1 m and 3 m).

FIGS. 23A and 23B are graphs showing impact of 1.5% cryoprotectant on 10 μm (FIG. 23A) and 25 μm (FIG. 23A) particles in DR formulations, at different temperatures (25° C. and 40° C.) and times (at to, 1 m and 3 m).

FIG. 24 is a graph depicting N₂ isotherm measured at 77 K as a function of relative pressure.

FIG. 25 is a graph depicting log differential pore size distribution data based on Hg intrusion volume.

FIG. 26 is a graph depicting cumulative pore size distribution data based on Hg intrusion volume.

### DETAILED DESCRIPTION OF THE INVENTIONS

Unless defined otherwise, all technical and scientific terms used herein have the same meaning as commonly understood by one of ordinary skill in the art to which this invention belongs.

### Definitions

The term “about” in the context of numerical values and ranges refers to values or ranges that approximate or are close to the recited values or ranges such that the invention can perform as intended, such as having a desired rate, amount, density, degree, increase, decrease, percentage, value or presence of a form, variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Antibodies” (also referred to as “immunoglobulins”) are examples of proteins having multiple polypeptide chains and extensive post-translational modifications. The canonical immunoglobulin protein (for example, IgG) comprises four polypeptide chains-two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014). Antibodies are often used as therapeutic biomolecules.

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10-9 M, at least 10-10 M; at least 10-11 M; or at least 10-12 M, as measured by surface plasmon resonance, for example, BIACORE™ or solution-affinity ELISA.

The antibodies can be based upon all major antibody classes, namely IgG, IgA, IgM, IgD and IgE. IgG is a preferred class, and includes subclasses IgG1 (including IgG1) and IgG1K), IgG2, IgG3, and IgG4. Antibodies include a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′) 2 fragment, an IgD antibody, an IgE antibody, an IgM antibody, an IgG antibody, an IgG1 antibody, an IgG2 antibody, an IgG3 antibody, or an IgG4 antibody.

The antibody can be an IgG1 antibody. The antibody can be an IgG2 antibody. The antibody can be an IgG3 antibody. The antibody is an IgG4 antibody. The antibody can be a chimeric IgG2/IgG4 antibody. The antibody can be a chimeric IgG2/IgG1 antibody. The antibody can be a chimeric IgG2/IgG1/IgG4 antibody. Derivatives, components, domains, chains and fragments of the above also are included.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, Fab, ScFv-Fc, dAB-Fc, half antibodies and other combinations of heavy and/or light chains), multispecifics (for example, bispecifics, IgG-ScFv, IgG-dab, ScFV-Fc-ScFV, trispecifics).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V2591), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

Some recombinant Fc-containing proteins contain receptors or receptor fragments, ligands or ligand fragments that have cognate binding partners in biological systems, and include “Receptor Fc-fusion proteins,” which refer to recombinant molecules that contain a soluble receptor fused to an immunoglobulin Fc domain.

“Fc-fusion proteins” comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci USA 88:10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-1R1 extracellular region fused to Fc of hIgG1; see U.S. Pat. No. 6,927,044, or a VEGF Trap (for example, Aflibercept, which contains the Ig domain 2 of the VEGF receptor Fltl fused to the Ig domain 3 of the VEGF receptor Flkl fused to Fc of hIgG1 See U.S. Pat. Nos. 7,087,411 and 7,279,159.

Rilonocept and aflibercept are examples of marketed trap proteins that antagonize IL1R (see U.S. Pat. No. 7,927,583) and VEGF (see U.S. Pat. No. 7,087,411), respectively. Other recombinant Fc-containing proteins include those recombinant proteins containing a peptide fused to an Fc domain. Recombinant Fc-containing proteins are described in C. Huang, “Receptor-Fc fusion therapeutics, traps, and MFMETIBODY technology,” 20 (6) Curr. Opin. Biotechnol. 692-9 (2009).

There also are proteins that lack Fc portions, such as recombinantly produced enzymes and mini-traps, also can be lyophilized according to the inventions. Mini-traps are trap proteins that use a multimerizing component (MC) instead of an Fc portion, and are disclosed in U.S. Pat. Nos. 7,279,159 and 7,087,411. Derivatives, components, domains, chains and fragments of the above also are included.

“Lyophilized cake” or “cake” refers to a lyophilized drug, and includes a protein drug in a lyophilized form in a container, such as a vial.

“Platform” refers to approaches in current use, and includes standard approaches.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein. All recited numbers or values expressly set forth and denominate all ranges created by the recited number or values

### DESCRIPTION

Lyophilization of biological products, such as Fc-containing proteins, has long been utilized for medicinal purposes. Lyophilized products tend to have longer shelf-lives than a liquid product due to the absence of water and decreased internal molecular dynamics. Additionally, lyophilized products can be reconstituted to a higher protein concentration than the concentration of the pre-lyophilization solution. Higher concentrations are useful for intravenous and subcutaneous administration. Moreover, subcutaneous administration requires a higher concentration of the biological products upon reconstitution.

Protein drug products are usually stored at 2 to 8° C. and room temperature stable (storage) will be a great improvement. Lyophilization manufacture process is usually time and energy intensive depending on the process design. A typical lyophilization process contains three sequential stages:
i. Freezing (may include annealing or controlled nucleation step);
ii. Primary drying (can it be designed to improve the process efficiency and protein storage stability); and
iii. Secondary drying (if is this stage necessary).

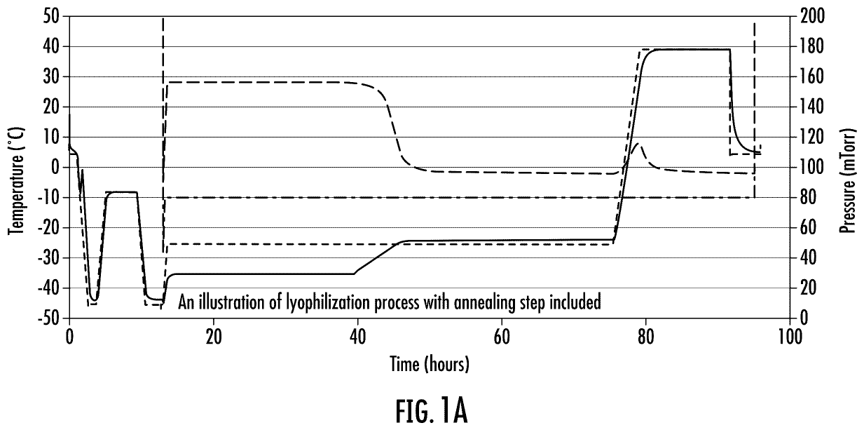

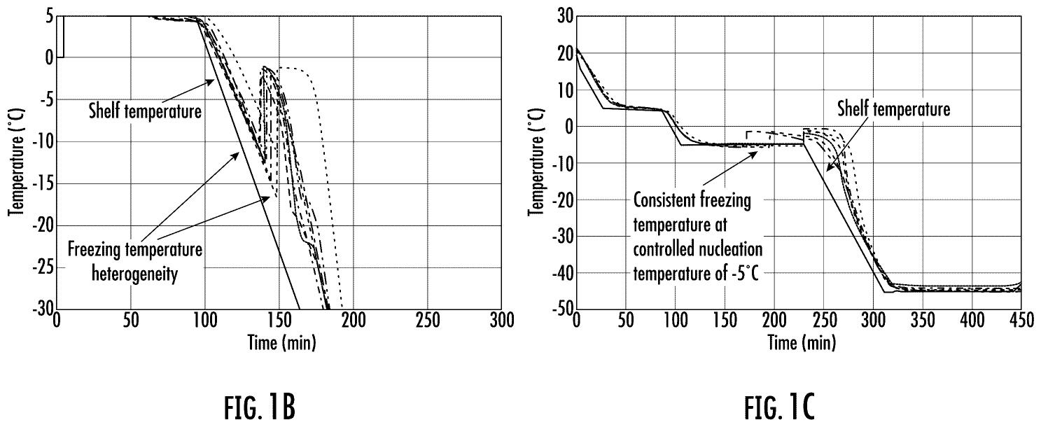

Primary drying is usually the longest phase and the focus for process efficiency. To achieve the goal of patient convenience, and energy, cost saving (as well as environment protection), improving stability and process efficiency are becoming the major emphasis. Illustration of a typical Lyophilization process with annealing step included in shown in FIG. 1A.

### Strategies to Improve Lyophilized Drug Products (DP)

Following strategies can be used:
1. Stabilizers such as sucrose (or trehalose) can be used in lyophilized formulations:
   (a) Sucrose and trehalose are disaccharides and are used as cryoprotectant and lyoprotectant. It can be challenging to have sufficient stabilizer to protein ratio for high concentration protein formulations;
   (b) Form an amorphous matrix to stabilize protein in the solid state as stabilizers are in the same phase as protein; and/or
   (c) Use water as an alternative stabilizer, which does not increase formulation viscosity/osmolality.
2. Formulation strategy to improve lyophilization process efficiency:
   (a) Use crystalline bulking agent to increase formulation collapse temperature (Tc). Suitable choices include mannitol and glycine (with high eutectic temperature, ˜33° C.); and/or
   (b) Lyophilization can be performed above Tg′, but below Tc to reduce the total ice sublimation time.
3. Formulation and/or process strategies to improve lyophilized DP:
   (a) Combinations of Stabilizers/Plasticizers (with smaller molecular weight including water molecules) can be use;
   (b) A stable protein with high water content may require less intensive or no secondary drying; and/or
   (c) Controlled nucleation and post lyophilization annealing can be used.

### Freeze-Drying Process Designs

Following conditions can be applied:
1. Freezing/cooling rate:
   (a) Fast freezing rate (e.g., 2° C./min), which usually not achievable at low temperature (e.g., <˜30° C.); and
   (b) Can be 0.5 to 1° C./min.
2. Freezing temperature (simple but critical):
   (a) Below Tg′ to ensure all solutes are in solid state; and
   (b) Below −35° C. because of product super-cooling (not scalable). Super-cooling is not a concern if controlled nucleation (CN) technology is used; and
   (c) Usually-40/−50° C.
3. Annealing for bulking agent or amorphous drug:
   (a) Crystallization of bulking agent;
   (b) Temperature between Teuand Tg′ with a duration 2-10 hours. Annealing at higher temperature requires shorter time; and
   (c) Improve amorphous drug cake morphology and drying efficiency

The experimental design used herein is as follows in Table 1 and associated text.

TABLE 1*

| Formulation Description | Formulation Composition |
|---|---|
| Platform Drug Product | 50 mg/ml mAb, 10 mM histidine, |
| | 5% sucrose, 0.1% PS20, pH 6.0 |
| Platform Placebo | 10 mM histidine, 5% sucrose, 0.1% PS20, |
| Product | pH 6.0 |
| Low Concentration | 2 mg/ml mAb, 10 mM histidine, |
| Drug Product | 10% sucrose, 0.1% PS20, pH 6.0 |
| Low Concentration | 10 mM histidine, 10% sucrose, |
| Placebo Product | 0.1% PS20, pH 6.0 |
| High mAb | 150 mg/ml mAb, 10 mM L-histidine, |
| Concentration and High | 5% w/v sucrose, 0.1% PS80, pH 6.0 |
| Sugar Concentration | |
| High mAb | 150 mg/ml mAb, 10 mM L-histidine, |
| Concentration and Low | 1.5% w/v sucrose, 0.1% PS80, pH 6.0 |
| Sugar Concentration | |
| Drug Product F1: | 150 mg/ml mAb3, 10 mM L-Histidine, |
| Conventional Cycle | 5% w/v sucrose, 0.1% w/v PS80, pH 6.0 |
| Drug Product F2: −5° C. | 150 mg/ml mAb3, 10 mM L-Histidine, |
| CN Cycle | 5% w/v sucrose, 0.1% w/v PS80, pH 6.0 |
| Drug Product F3: | 150 mg/mL mAb3, 10 mM L-Histidine, |
| Conventional Cycle | 1.5% w/v sucrose, 0.1% w/v PS80, pH 6.0 |
| Drug Product F4: −5° C. | 150 mg/ml mAb3, 10 mM L-Histidine, |
| CN Cycle | 1.5% w/v sucrose, 0.1% w/v PS80, pH 6.0 |

*20R with 5.3 mL fill for each formulation. (5.3 mL in 20 mL type 1 glass vials, lyo DP reconstituted with 4.56 mL WFI to 150 mg/ml formulations). Purpose of the experiment was to evaluate the impacts of CN Lyo Cycle on the mAb3 Drug Products stability.

### Lyophilization Cycle Types:

(a) Platform placebo lyophilization cycle (−20° C. for 50 hours and 40° C. for 12 hours), which is a conventional lyophilization method.
(b) Anneal with a conventional lyophilization cycle with annealing (−5° C. for 6 hour). Use a conventional lyophilization method, such as the platform placebo lyophilization cycle, preceded by annealing.
(c) Lyophilization cycle with controlled nucleation (CN) at −5° C.
(d) Lyophilization cycle with controlled nucleation (CN) at −10° C.

### Assays:

Typical analytical assays for lyophilized product include visual, size exclusion chromatography (SEC), cation exchange chromatography (CEX) and micro-flow imaging (MFI).

Lyophilization process usually has three stages: (1) Freezing, (2) Primary drying by sublimation and (3) Secondary drying by desorption. The freezing step is the first part of lyophilization. In conventional platform approaches, freezing can occur randomly. See FIG. 1B. Conventional platform techniques yield lower freezing temperatures (more supercool), such as −11° C. to −17° C., which results in substantial super cooling and freezing temperature heterogeneity, as shown in FIG. 1B.

Controlled nucleation freezing is less supercool with consistent temperature (e.g., −5° C.). FIG. 1C shows controlled nucleation taking place at −5° C. according to the inventions. The arrow in FIG. 1C points to the consistent freezing temperature, and stands in stark contrast to the heterogeneous temperatures shown in FIG. 1B. Temperatures no lower than −10° C. are preferred, such as about −2° C., −2.5° C., −3° C., −3.5° C., −4° C., −4.5° C., −5° C., −5.5° C., −6° C., −6.5° C., −7° C., −7.5° C., −8° C., −8.5° C., −9° C., −9.5° C., or −10° C., as well as additional subranges made based upon the above values.

There are various methods to achieve controlled nucleation, including (1) Seeding and vial pre-treatment, such as using of additives/surface roughening, (2) ultrasonic-nucleation: short vibration, (3) use of ice fog by introduction of cold nitrogen gas into the chamber to freeze out moisture within the chamber and (4) depressurization, but at the risk of product splash.

The preferred approach according to the invention employs pressurization, such as 14 to 42 psig, preferably 25 to 30 psig, and more preferably about 28 psig. Pressure in the lyophilization chamber can be determined using a Pirani gauge, for example. The basic approach includes:
(1) Cool the product to intended freezing temperature (slightly below its freezing temperature);
(2) Pressurize the lyophilization chamber with air or an inert gas, such as nitrogen or argon (about 28 psig); and
(3) Release the pressure quickly to introduce ice nuclei.

More specifically, the inventions provide for an implementation can include:
Loading containers and seal freeze-dryer;
Cooling shelf and containers to target nucleation temperature;
Pressurizing with air or an inert gas (for example, helium, nitrogen and/or argon);
Depressuring quickly to induce nucleation; and
Reducing shelf temperature to complete freezing.

Typically, The present inventions provide improved methods of lyophilization that yield products with improved properties, as described herein. The inventions employ controlled nucleation.

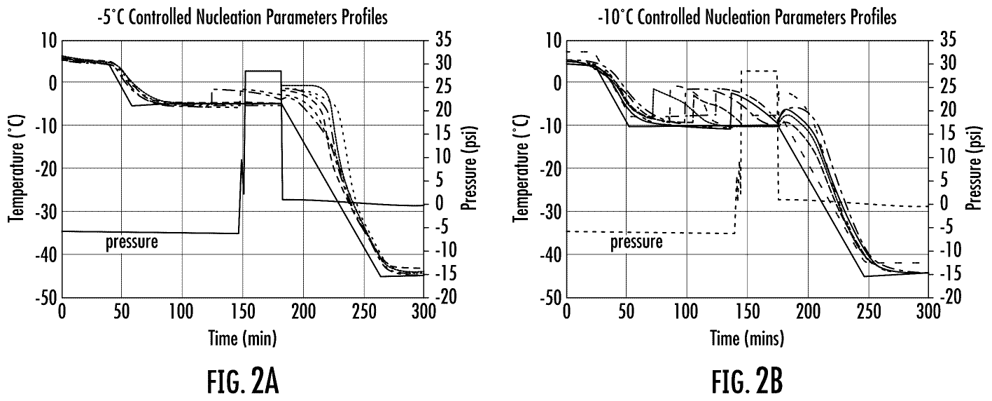

FIGS. 2A and 2B depict data from controlled nucleation parameters profiles of different monoclonal antibody drug products preparation. FIG. 2A is at −5° C. and FIG. 2B is at −10° C. Controlled nucleation at −5° C. ensured ice formation for all the vials at the set temperature with less supercooling than compared −10° C. controlled nucleation. For −5° C., approximately a 30 minute holding time is sufficient during the nucleation. See FIG. 2A. Holding times can be 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20 minutes, or times longer or shorter, as determined in view of the teachings contained in herein.

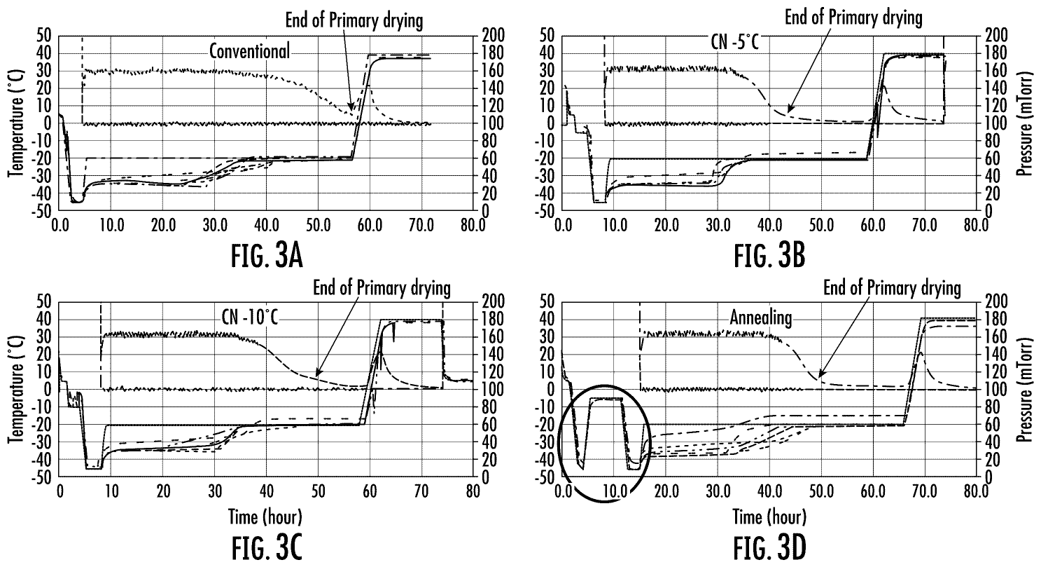

FIGS. 3A to 3D depict data from various lyophilization approaches on different monoclonal antibody drug product preparation. FIG. 3A depicts data from a conventional platform lyophilization. FIG. 3B depicts data from controlled nucleation at −5° C. FIG. 3C depicts data from a controlled nucleation at −10° C. FIG. 3D depicts data from a conventional platform lyophilization with annealing (see encircled between 0.0 and about 15 hours. Arrows in each figure indicate the end of primary drying.

Table 2 below depicts data that shows that lower product resistance for ice sublimation from controlled nucleation (CN) and annealing cycles as compared to conventional platform.

TABLE 2

| Lyophilization | Con- | CN at | CN at | |
|---|---|---|---|---|
| Cycles | ventional | −5° C. | −10° C. | Annealing |
| Tp (° C.) | −34.5 | −35 | −35 | −35 |
| Pice (Torr) | 0.177 | 0.168 | 0.168 | 0.168 |
| Primary drying | 51.6 | 33 | 40.6 | 33.3 |
| time (hour) | | | | |
| Ice sublimation | 0.10 | 0.15 | 0.12 | 0.15 |
| rate (g/h) | | | | |
| Normalized | 4.8 | 2.7 | 3.3 | 2.7 |
| resistance | | | | |
| (Torr · h · cm2/g) | | | | |

$$R \hat{} p = Rp \cdot Ap \qquad {R}_{p} + {R}_{s} = \frac{{P}_{ice} - {P}_{c}}{\frac{Δ m}{Δ t}} \qquad {P}_{ice} = {e}^{\frac{- 6144.96}{T} + 24.01849}$$

Where:
R \hat{ } p is the area normalized product resistance (Torr·h·cm²/g) and Rp is resistance (Torr·h/g);
Ap is the cross-sectional area of the product (cm²);
Rp and Rs are the dry layer and stopper resistance, respectively, to water vapor transport from the sublimation interface (Torr·h/g);
Δm/Δt is the average ice sublimation rate (g/hour per vial);
Pice is the equilibrium vapor pressure of ice at the sublimation interface temperature (Torr);
PC is the chamber pressure; and
TP is the product temperature at sublimation interface (K).

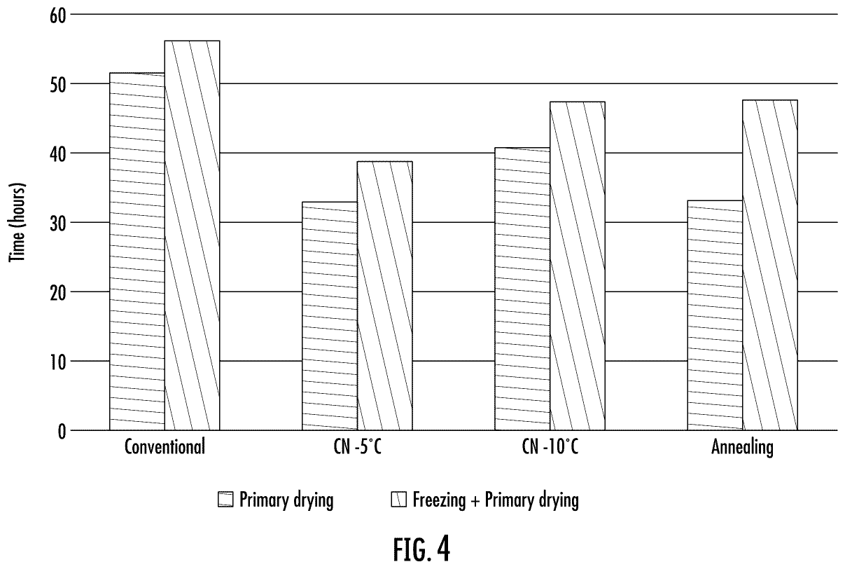

FIG. 4 depicts primary drying times and freezing plus primary drying times for conventional platform (see FIG. 3A), controlled nucleation at −5° C. (see FIG. 3C), controlled nucleation at −10° C. (see FIG. 3C), and conventional platform with annealing (see FIG. 3D).

Overall, controlled nucleation significantly reduced lyophilization cycle time. Controlled nucleation at −5° C. was faster than controlled nucleation at −10° C. Annealing also reduced the primary drying time, but the annealing added extra cycle time compared to controlled nucleation at −5° C. Freezing plus primary drying for controlled nucleation at −5° C. overall took about 39 hours, whereas the annealing approach took about 48 hours overall.

Next, moisture content evaluation for conventional platform, annealing, controlled nucleation (CN) at −5° C. (performed twice) and controlled nucleation at −10° C. (performed twice) lyophilizations were undertaken and the results set are set forth in Table 3.

TABLE 3
% Moisture Content (t = 0 samples)

| | Platform | Platform | Low Conc. DP | Low |
|---|---|---|---|---|
| | DP* | PP** | (2 mg/ml mAb | Conc. PP |
| Lyophilization | For- | For- | and 10% sucrose) | (10% sucrose) |
| Approach | mulation | mulation | Formulation | Formulation |
| Conventional | 0.2% | 0.2% | 0.6% | 0.6% |
| Annealing | 0.4% | 0.8% | 1.1% | 0.7% |
| CN −5° C. | 0.4% | 0.4% | 1.2% | 0.8% |
| CN −5° C. Repeat | 0.5% | 0.8% | 1.3% | 1.0% |
| CN −10° C. | 0.6% | 0.3% | 0.9% | 0.5% |
| CN −10° C. | 0.3% | 0.4% | 1.0% | 0.7% |
| Repeat | | | | |

*Platform DP = 5.3 mL fill of Platform formulation (50 mg/ml mAb, 10 mM histidine, 5% sucrose, 0.1% PS20, pH 6.0) in 20R vial.
*Platform PP = 5.3 mL fill of Platform matching placebo formulation in 20R vial.

All approaches create lyophilized products with acceptable moisture contents (less than 4%). Increased moisture contents were expected from annealing or controlled nucleation lyophilization cycles. More aggressive secondary drying conditions should be used when using controlled nucleation if lower moisture content is required.

Reconstitution Times for Lyophilized Drug Product Manufactured with Conventional Platform, Annealing and Controlled Nucleation lyophilization approaches were then assessed, and the results are set forth in Table 4 below.

TABLE 4
Reconstitution Time (Minutes:Seconds)

| | Drug Product | Drug Product |
|---|---|---|
| | reconstituted to | reconstituted to |
| Lyophilization approach | 50 mg/ml | 150 mg/ml |
| Conventional | 1:18 | 8:40 |
| Annealing | 1:01 | 3:35 |
| CN −5° C. | 0:46 | 2:05 |
| CN −5° C. Repeat | 0:49 | 1:45 |
| CN −10° C. | 0:56 | 6:05 |
| CN −10° C. Repeat | 0:59 | 6:57 |

The fastest reconstitution time when reconstituted to a high concentration antibody drug product (150 mg/ml) was observed with controlled nucleation (CN)−5° C. The reconstitution time can be less than 3 minutes. More particularly, the reconstitution time can be about or less than 2 minutes and 50 seconds, 2 minutes and 40 seconds, 2 minutes and 30 seconds, 2 minutes and 20 seconds, 2 minutes and 10 seconds, 2 minutes, 1 minute and 50 seconds, one minute and 45 seconds, 1 minute and 40 seconds or less. Reconstitution to a lower concentration is even faster. For example, reconstitution time for a 50 mg/ml antibody drug product can be about or less than 59, 58, 57, 56, 55, 54, 53, 52, 51, 50, 49, 48, 47, 46, 45, 44, 43, 42, 41, 40 seconds or less.

The slowest approach was conventional platform lyophilization. A similar, but less pronounced, data trend was shown at a reconstitution to 50 g/ml. A very low concentration drug product reconstituted to 2 or 6 mg/ml, and a placebo product showed no appreciable differences in reconstitution time (all less than 1 minute) (data not shown).

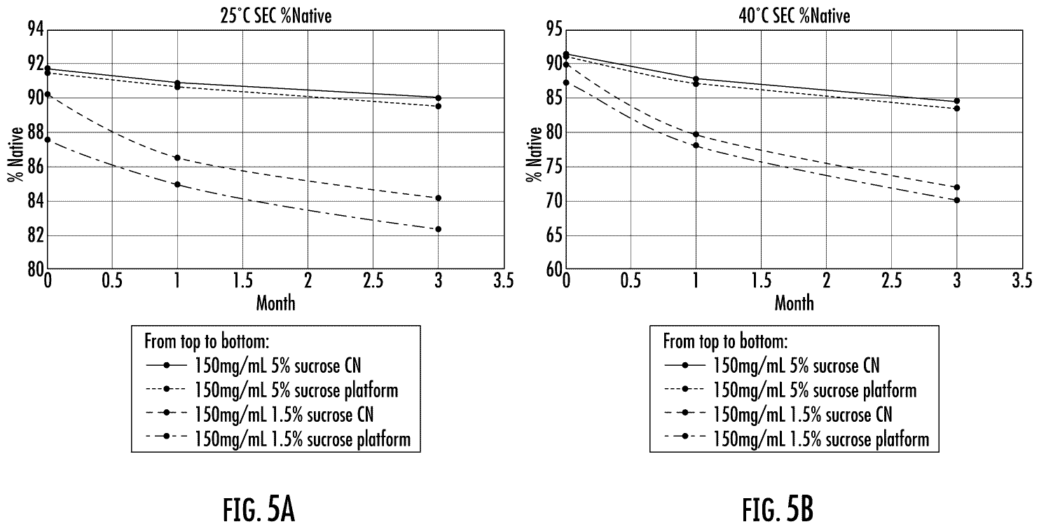

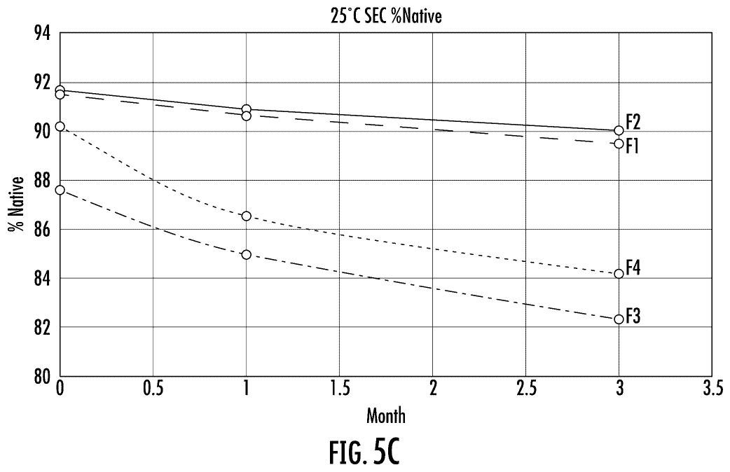

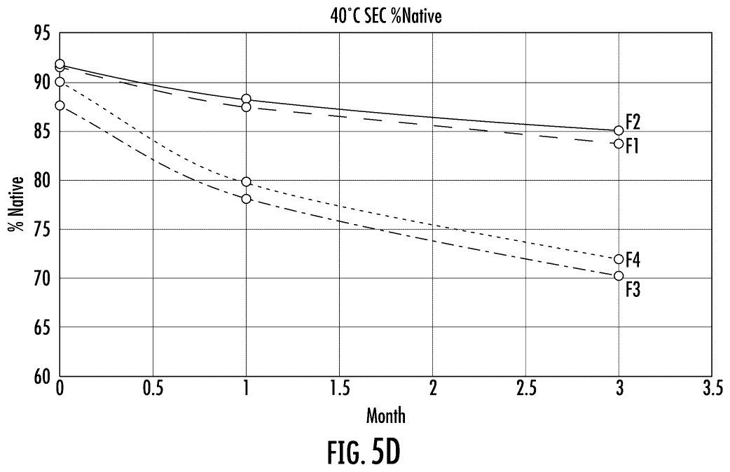

Effects of cryoprotectant and formation of high molecular weight (HMW) aggregates in samples stored at 25° C. and 40° C. were analyzed using size exclusion chromatography (SEC). FIGS. 5A and 5B depict SEC data for percent native mAb using buffers comprising 150 mg/ml mAb and 5% sucrose or 1.5% sucrose (See Table 1). FIG. 5A is for formulations stored at 25° C. and FIG. 5B is for formulations stored at 40° C. FIGS. 5C and 5D depict SEC and high molecular weight (HMW) data for formulations F1, F2, F3, and F4 (See formulations in Table 1). FIG. 5C is for formulations stored at 25° C. and FIG. 5D is for formulations stored at 40° C. Percent (%) protein purity was found to be higher in post-lyophilization (less HMW formation) with CN vs conventional lyophilization cycle for formulation with 1.5% sucrose (F4 vs F3). The rate of degradation are comparable. Slightly better stability was observed comparing CN (F2) to conventional (F1) for stable formulation with 5% sucrose.

Each mAb formulation (5% sucrose and 1.5% sucrose) was lyophilized a using a conventional platform approach and controlled nucleation. The formulations were stored at 25° C. and 40° C. prior to the SEC.

For both 25° C. and 40° C. storage, there was little difference in molecular weight curves between conventional and controlled nucleation for the 5% sucrose formulations. In contrast, 1.5% sucrose formulations stored at 25° C. and 40° C. showed degradation over time. Controlled nucleation lyophilization exhibited less degradation than conventional lyophilization at both temperatures using the 1.5% sucrose formulation.

In the comparisons above, for both controlled nucleation lyophilization and conventional lyophilization and storage at both temperatures, there was no meaningful difference in protein recovery. Protein recovery ranged from 97% to 99%.

As there is a need for a more stable drug product using more efficient lyophilization process, advances in formulation and process strategies can be applied to improve lyophilized protein drug products.

The inventions provide that increasing lyophilized drug moisture content can be an efficient way of improving DP stability. Water appeared to be the most effective plasticizer for reducing local mobility of mAb molecules and the target optimum moisture content can be achieved by lyophilization process control.

The inventions also provide a lyophilized protein drug product that is stable at room temperature and manufactured with efficient lyophilization process.

The inventions are further described by the following examples, which do not limit the inventions in any manner. The order of performance of the below experiments and/or examples or example steps can be altered or combined as determined by the person of skill in the art in view of the teachings and data contained herein.

### Example 1-Lyophilized mAb Cakes in Vials

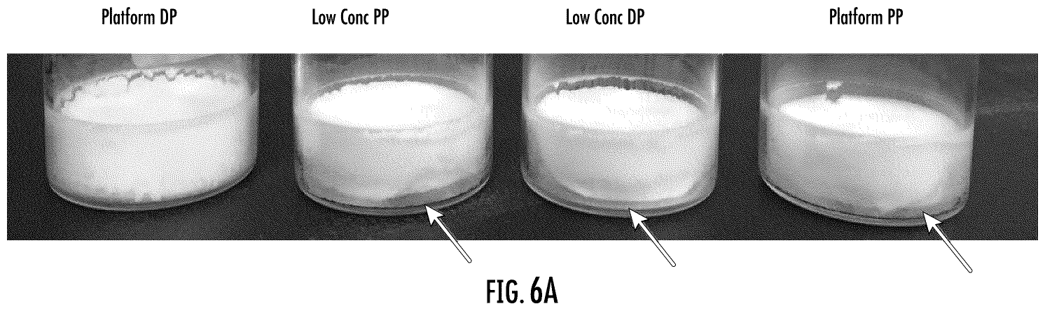

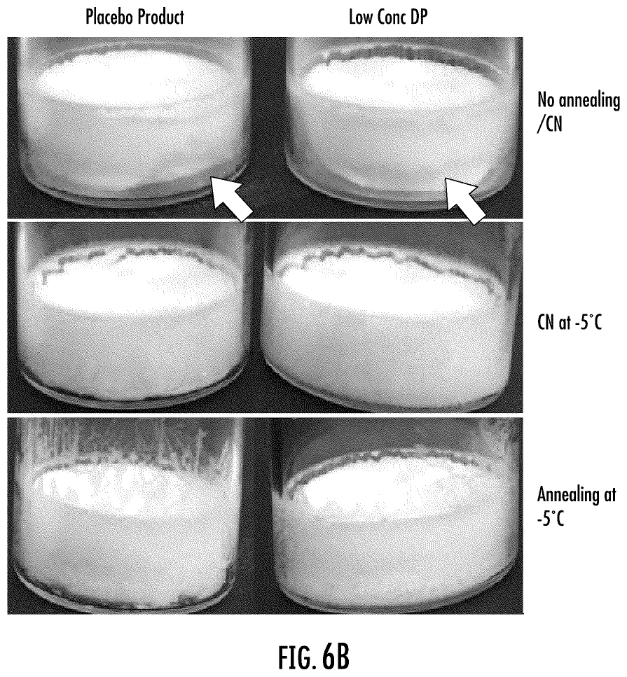

FIG. 6A shows lyophilized mAbs preparations in vials: Platform Drug Product (DP), Low concentration Placebo Product (PP), Low concentration Drug Product and Platform Placebo Product from left to right. The formulations are set forth in Table 1.

The lyophilized mAbs in three of the four vials at the bottom showed product shrinkage at the bottom of the vial, designated by arrows. If upon visual inspection the shrinkage is deemed to be caused by cake collapse or melt back, the vials would be considered unacceptable.

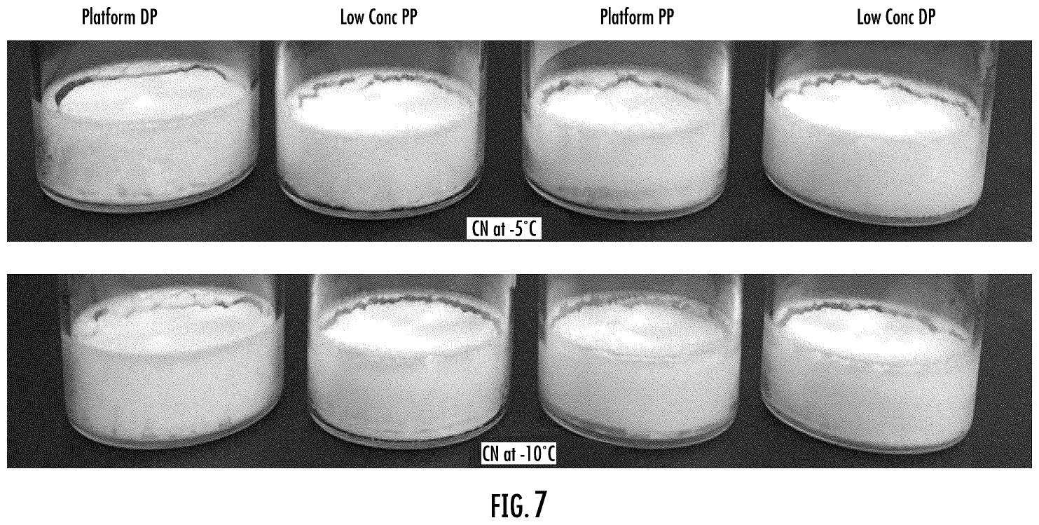

FIG. 7 shows lyophilized mAbs preparations in vial prepared using controlled nucleation: Platform Drug Product, Low concentration Placebo Product, Platform Placebo Product and Low concentration Drug Product from left to right. The top row was performed at −5° C. and the bottom row was performed at −10° C. See formulations in Table 1. Overall, the lyophilized mAbs with controlled nucleation showed no apparent shrinkage as compared to the conventional lyophilization of mAbs shown in FIG. 6A.

Lyophilized mAbs preparations in vials are depicted in FIG. 6B. Placebo drug product and low concentration drug product (DP) using lyophilization with annealing at −5° C. (bottom row), with no annealing controlled nucleation (top row), and controlled nucleation at −5° C. (middle row). The arrows in the figures indicate product shrinkage. Although both controlled nucleation and annealing can improve cake structure, annealing may cause other unexpected issues, such as:
(a) Potential product on the wall, and/or
(b) skin formation resulting in cake collapse.

Moderate product shrinkage is typically acceptable (product is not acceptable if treated as cake collapse or melt back by visual inspection).

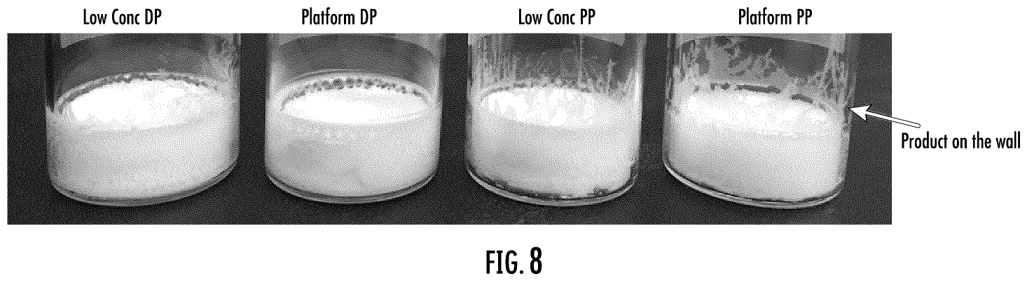

FIG. 8 shows lyophilized mAbs preparations in vials with annealing performed prior to lyophilization: Low concentration Drug Product (DP), Platform Drug Product, Low concentration Placebo Product (PP), and Platform Placebo Product from left to right. See formulations in Table 1.

The addition of the annealing step to the lyophilization reduced cake shrinkage but also resulted in mAb product adhering to the vial wall (see arrow in FIG. 8).

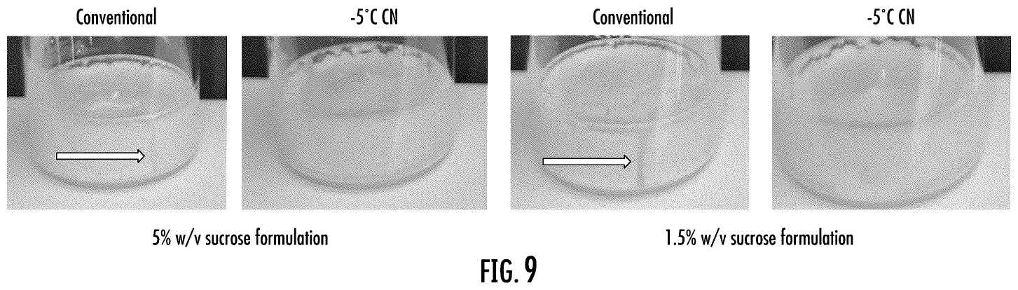

FIG. 9 shows Platform lyophilized high concentration (150 mg/mL) mAb preparations and lyophilized high concentration (150 mg/mL) mAb preparations in vials made with controlled nucleation at −5° C. using the 5% w/v sucrose formulation (left) and the 1.5% sucrose formulation (right). The lyophilized mAb cake with controlled nucleation exhibited less cracking than the conventionally lyophilized mAb cake. See formulations in Table 1. The arrows in FIG. 9 indicate cracking in the conventionally lyophilized mAb cake.

Cake appearance improvement: DP from −5° C. CN showed less or no cracks in the lyo cakes.

Product purity: CN showed higher purity (less aggregation) when compared to conventional (for formulation of 150 mg/ml mAb3 with 1.5% v/w sucrose).

No substantial change was observed in CEX and particles at 25° C. and 40° C. in three months.

### Example 2-Electron Microscopy of Lyophilized mAbs

Lyophlized mAb samples were subjected to scanning electron microscopy (SEM) to assess morphology. 100 μm size bars are located in the upper right corner of each figure.

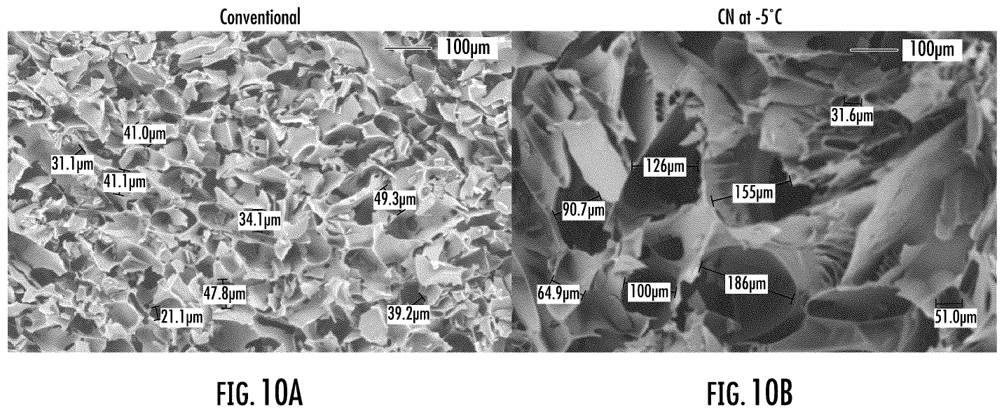

FIGS. 10A and 10B show scanning electron micrographs at the same scale. FIG. 10A is from a platform lyophilized preparation (50 mg/ml) and FIG. 10B is from a lyophilized preparation made with controlled nucleation at −5° C. See formulations in Table 1.

### Cake Morphologies (SEM) Comparison Between Conventional and Controlled Nucleation Lyophilization Cycles:

Conventional nucleation lyophilization (FIG. 10B) exhibited larger pore size than the conventional lyophilization (FIG. 10A). The larger pore size provided by controlled nucleation results in faster reconstitution time and smaller cake resistance during ice sublimation.

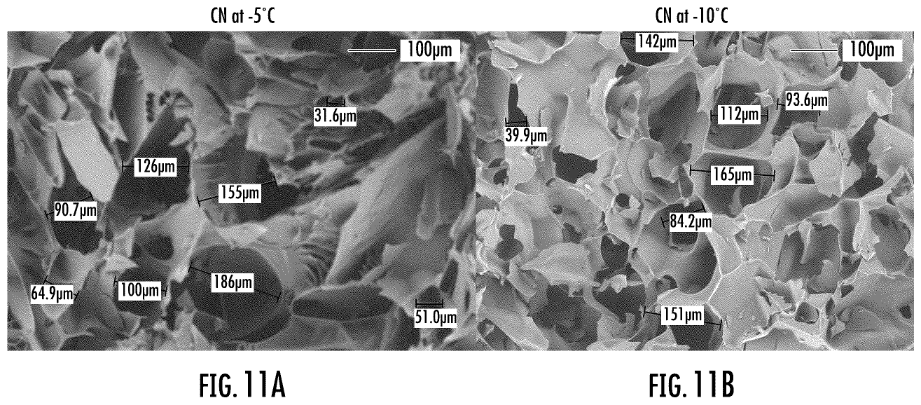

FIGS. 11A and 11B show scanning electron micrographs at the same scale. FIG. 11A is from a lyophilized platform (50 mg/ml) preparation made with controlled nucleation at −5° C., and FIG. 11B is from a lyophilized platform (50 mg/ml) preparation made with controlled nucleation at −10° C. See formulations in Table 1. Controlled nucleation at −5° C. (FIG. 11A) provides a larger pore size than controlled nucleation at −10° C. (FIG. 11B), which results in faster reconstitution time and smaller cake resistance during ice sublimation. FIG. 11A is the same as FIG. 10B. The micrograph was repeated to facilitate comparisons.

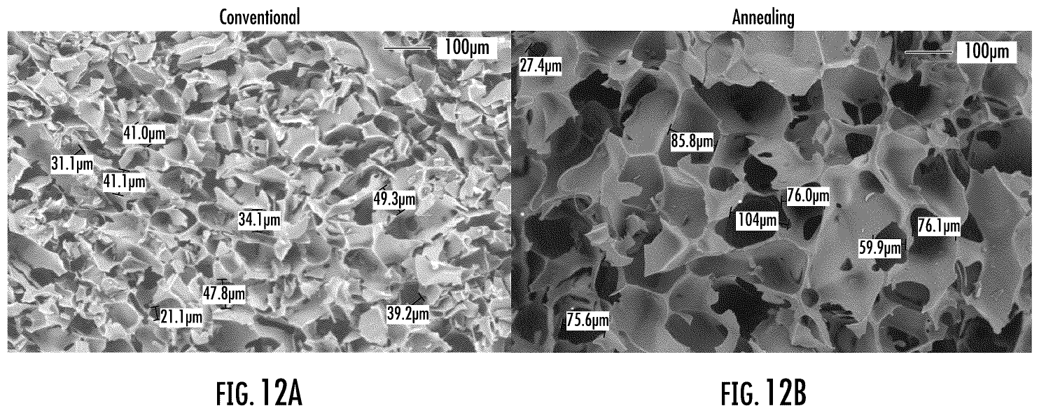

FIGS. 12A and 12B show scanning electron micrographs at the same scale. FIG. 12A is from a conventional platform lyophilization. FIG. 12B is from a conventional platform lyophilization with annealing. See formulations in Table 1. The addition of the annealing step allows convention lyophilization to provide larger pores. However, as indicated by the 100 μm size bars, the pores in both FIGS. 12A and 12B are smaller than the pores in FIGS. 10B and 11A. Again, larger pore size from controlled nucleation at −5° C. results in faster reconstitution time (see Table 4) and smaller cake resistance during ice sublimation. Additionally, as shown in FIG. 4, annealing takes longer time than controlled nucleation at −5° C.

Different cake morphologies with drastic larger pore size for drug products produced from controlled nucleation and annealing lyophilization cycles can be revealed using SEM. SEM micrographs show that larger pore size from CN at −5° C. than from CN at −10° C. (see FIG. 10B v. 11B). SEM micrographs further reveal that annealing also increased pore size significantly (see FIG. 12B). Larger pore size result in smaller cake resistance and faster ice sublimation, therefore shorter primary drying time.

### Example 3—The Effects of Annealing

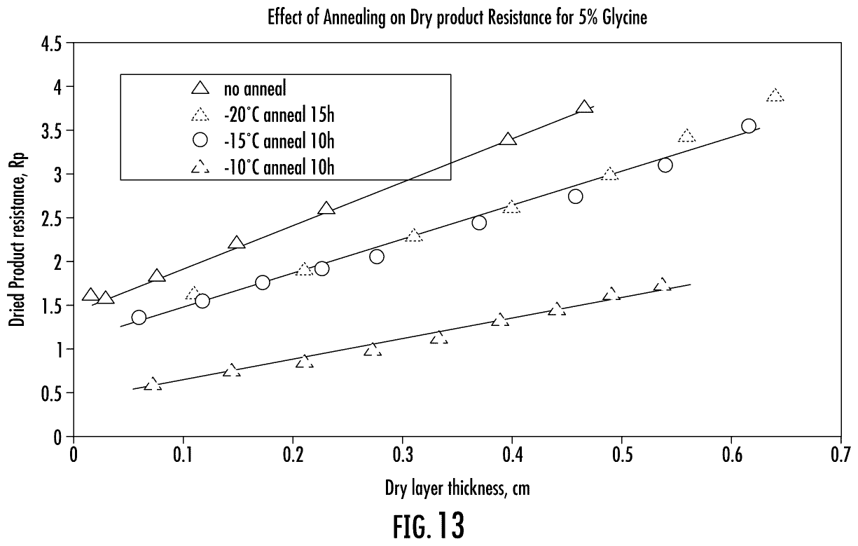

Effect of annealing on dry product resistance for 5% glycine is depicted in FIG. 13. The effects of annealing Include the following:
(a) Annealing reduces the dry product resistance and primary drying time;
(b) Annealing increases ice crystal size, and pore size in dry cake for ice sublimation;
(c) Higher annealing temperature is more effective; and
(d) Longer time at lower temperature can achieve the same produce resistance.

### Example 4-Exemplary Antibodies for Controlled Nucleation Lyophilization

According to additional aspects, the antibody can be selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-DII4 antibody, an anti-Angiopoetin-2 antibody (for example an anti-ANG2 antibody as described in U.S. Pat. No. 9,402,898), an anti-Angiopoetin-Like 3 antibody (for example an anti-AngPtl3 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-C5 antibody as described in U.S. Pat. AppIn. Pub. No US2015/0313194A1), an anti-TNF antibody, an anti-epidermal growth factor receptor antibody (for example an anti-EGFR antibody as described in U.S. Pat. No. 9,132,192 or an anti-EGFRvill antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat. No. 8,871,209 or U.S. Pat. No. 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g an anti-IL4R antibody as described in U.S. Pat. Appln. Pub. No. US2014/0271681A1 or U.S. Pat. No. 8,735,095 or U.S. Pat. No. 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088295A1 and US20150266966A1, and in U.S. Application No. 62/222,605), an anti-Cluster of differentiation 20 (for example an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation 48 (for example anti-CD48 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. AppIn. Pub. No. US2016/0017029 and U.S. Pat. Nos. 8,309,088, 9,353,176) and an anti-Activin A antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (for example, an anti-CD3×anti-Muc16 bispecific antibody), and an anti-CD3×anti-Prostate-specific membrane antigen bispecific antibody (for example, an anti-CD3×anti-PSMA bispecific antibody). See also U.S. Patent Publication No. US 2019/0285580 A1. Also included are a Met ×Met antibody, an agonist antibody to NPR1, an LEPR agonist antibody, a BCMA ×CD3 antibody, a MUC16×CD28 antibody, a GITR antibody, an IL-2Rg antibody, an EGFR ×CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured according to the inventions. Exemplary antibodies include Alirocumab, Atoltivimab, Maftivimab, Odesivimab, Odesivivmab-ebgn, Casirivimab, Imdevimab, Cemiplimab and Cemiplimab-rwlc (human IgG4 monoclonal antibody that binds PD-1), Dupilumab (human monoclonal antibody of the IgG4 subclass that binds to the IL-4R alpha (a) subunit and thereby inhibits Interleukin 4 (IL-4) and Interleukin 13 (IL-13) signalling), Evinacumab, Evinacumab-dgnb, Fasinumab, Fianlimab, Garetosmab, Itepekimab Nesvacumab, Odrononextamab, Pozelimab, Sarilumab, Trevogrumab, and Rinucumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

### Example 5-Primary Drying Design: Target Product Temperature and Chamber Pressure

As a general rule, the target product temperature should be at a “safety margin” below the collapse temperature.
a. Target Product Temperature is selected based on the following:
   i. Target Tp=Tc-(safety margin); and
   ii. Target Tp can be significantly above Tg′ (esp. for high concentration protein drug products).
b. Chamber pressure selection (calculation) is done based on the following:
   i. By allowing sufficient vacuum to drive ice sublimation (the lower chamber pressure the better);
   ii. By improving the heat transfer homogeneity (optimum ˜150-200 mT); and
   iii. Chamber pressure (Pc) is dependent upon target product temperature, as per the following equation:

$${P}_{c} = 0.29 \cdot {10}^{( 0.019 \cdot {T}_{p} )}$$

c. Shelf temperature and duration combination can be designed to control product moisture content:
   i. When a known moderate moisture content is desired.

### Example 6-Primary Drying Conditions for High Concentration Protein Formulations

150 mg/ml mAb formulations can be used with the Tc, which can be 15° to 20° C. above Tg′:
i. Strong glass formed with high concentration mAb; and
ii. Less difference between Tc and Tg′ is observed since the formulations are dominated by fragile glass formers.

Lyophilization can be performed at high shelf temperature for high concentration mAb formulations with product temperature above Tg′ (but below Tc)
i. Observed no lyophilized DP cake collapse

Conditions with several stabilizers are listed in Table 5.

Table 5 shows data from drying conditions for protein formulations with several stabilizers.

TABLE 5

| | Tc (partial), ° C. | Tg′, ° C. | Tc (° C.) | Tg′ (° C.) |
|---|---|---|---|---|
| Stabilizers | 2 mg/mL | | 150 mg/mL | |
| Sucrose | −28.0 (−32.2) | −34.1 | −3.5 | −24.2 |
| Trehalose | −28.5 (−30.5) | −31.3 | −5 | −22.6 |
| Arginine | −39.0 (−43.0) | −48.0 | 0.0 | −23.6 |
| Sorbitol | −39.1 (−43.5) | −46.7 | −2.5 | −28.6 |
| Glycerol | −59.2 (−64.0) | −65.0 | −13.5 | −30.2 |

### Example 7-Secondary Drying Design

Following criteria were considered for designing the secondary drying:
a. Chamber pressure
   i. Keep the same chamber as primary drying
   ii. Chamber pressure has little impact on water desorption rate (at or below 200 mT)
b. Shelf temperature and ramping rate
   i. “High” temperature required for water desorption;
      1. Below glass transition temperature,
   ii. Longer time needed at lower shelf temperature;
   iii. Potential over dry at high shelf temperature (esp. not enough stabilizer in formulation); and
   iv. Slow ramp to ensure desorption before the product at high temperature;
      1. More critical to amorphous product.
c. Duration
   i. Generally, several hours;
      1. Too short 2nd drying time can be difficult for scale up (moisture content heterogeneity), and
   ii. Avoid long duration for secondly drying (e.g., >12 hours), using allowable higher temperature for efficiency.
d. Lyophilization without secondary drying
   i. when moderate moisture content has advantage over “dry”.

### Example 8-Water as Plasticizer to Stabilize Protein at Moderate Moisture Content

Isotonic formulation of mAb (150 mg/mL) were subjected to various moisture contents at different storage temperature. Changes in % HMW aggregates were determined.

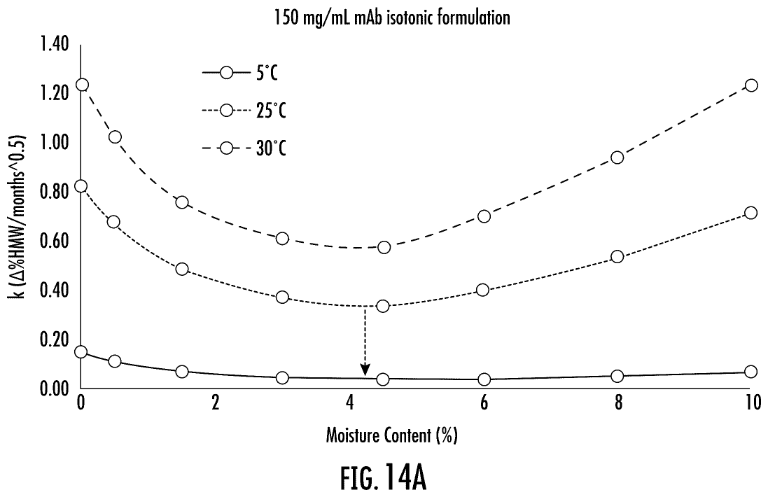

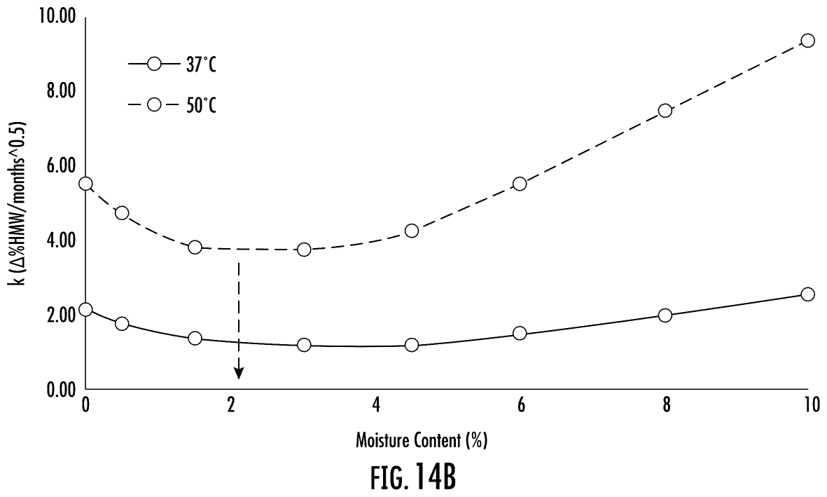

Graphs depicting the moisture content and stabilization of drug formulation at 5° C., 25° C. and 30° C. (FIG. 14A) and at 37° C. and 50° C. (FIG. 14B).

The lowest moisture content did not result in the best stability at storage conditions ranging from 5° C. to 50° C. In contrast, moderate moisture content of about 4% (about 2% to 5%) found to be optimal for room temperature storage. Optimal moisture content is lower at higher storage temperature (about 2% at 50° C.) (See FIG. 14B). There was no disadvantage of increasing formulation viscosity or osmolality.

### Example 9-Efficient Lyophilization Process to Achieve Moderate Moisture Content

Experiments were carried out to figure out an efficient lyophilization process in order to determine a moderate moisture content in the drug product.

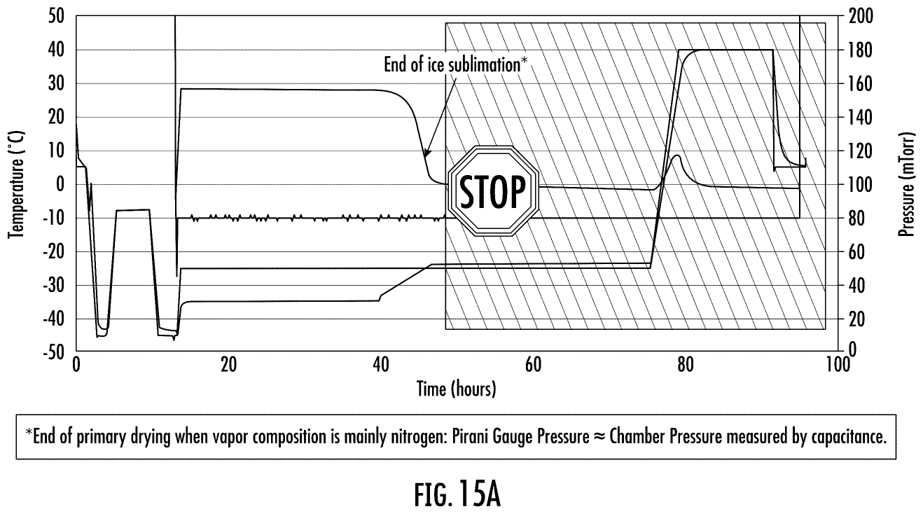

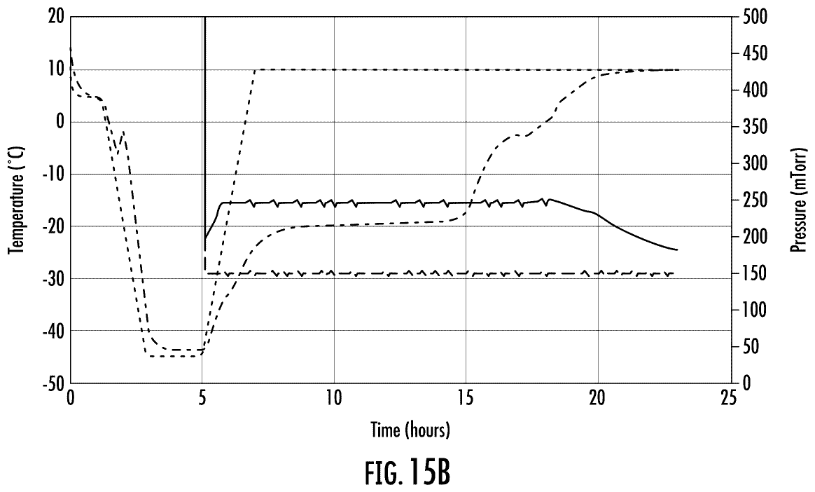

FIGS. 15A and 15B depict the results of efficient lyophilization process and the moderate moisture content in the DP. FIG. 15A indicates STOP following the end of ice sublimation.

A lyophilization cycle without secondary drying step was used to control the product target residual moisture content (e.g., about 2% to 4% residual moisture content)

A two-step lyophilization cycle: freezing and drying appeared to be a shorter process.

Moisture content can be controlled by drying shelf temperature (Ts) and duration post ice sublimation (without secondary drying step).

### Example 10-Optimum Combination of Shelf Temperature and Post Ice Sublimation Time

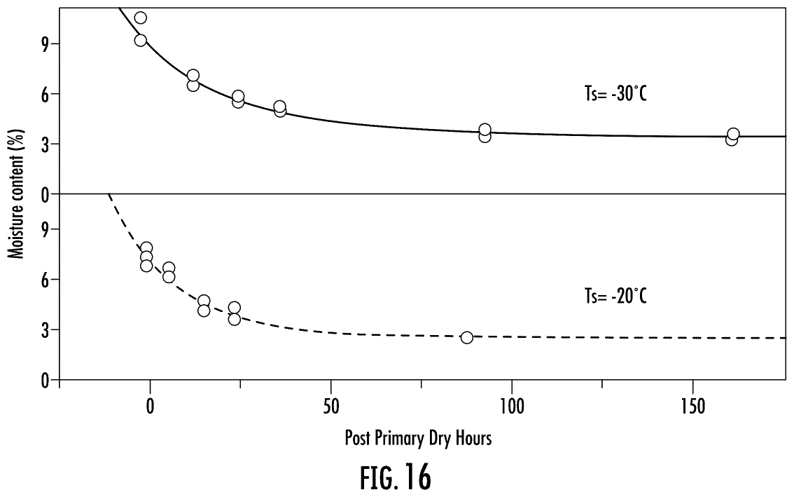

DP water content (after ice sublimation is complete) was controlled by combination of shelf temperature and duration of post primary drying time. FIG. 16 depicts the optimal combination of shelf temperature and post ice sublimation time for achieving target residual moisture content. Exponential decay fitting:

% H₂O−30° C.=5.59e−0.0388*t+3.36%

% H₂O−20° C.=4.72e−0.0528*t+2.42

The optimum combination of shelf temperature and post ice sublimation time were obtained by (a) controlling of DP water contend (after ice sublimation is complete) with combination of shelf temperature and duration of post primary drying. Extending primary drying time to achieve target residual moisture content; and (b) minimize the extending time to maximize the process efficiency, with change in shelf temperature as needed.

### Example 11-Room Temperature Stable Lyophilized Drug Product with Moderate Moisture Content

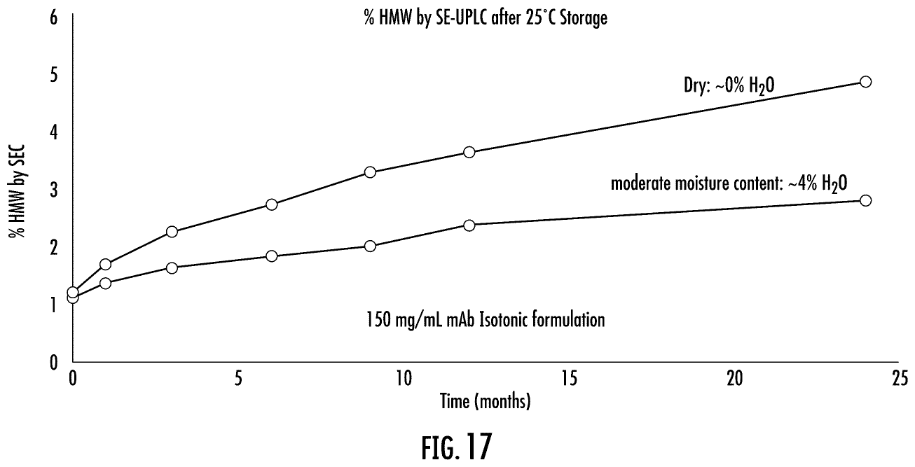

Isotonic formulation of mAb (150 mg/mL) were stored at about 0% and about 4% moisture contents at room temperature (about 25° C.). Changes in % HMW aggregates were determined by SEC-UPLC. FIG. 17 depicts % HMW, as determined by SE-UPLC, in stable lyophilized DP stored at 25° C. with moderate moisture content.

Monoclonal Ab appeared to be more stable with moderate (about 4%) moisture content when stored at room temperature (about 25° C.).

Room temperature stable lyophilized DP with optimal moisture content (about 4% H₂O). A high concentration (150 mg/mL) Isotonic formulation, which is suitable for IV and SC administration, found to be stable at room temperature when the DP formulation is stored lyophilized with the determined optimal moisture content (about 4% moisture content).

### Example 12-Results of Analytical Assays Using Size Exclusion Chromatography (SEC), Cation Exchange Chromatography (CEX) and Micro-Flow Imaging (MFI)

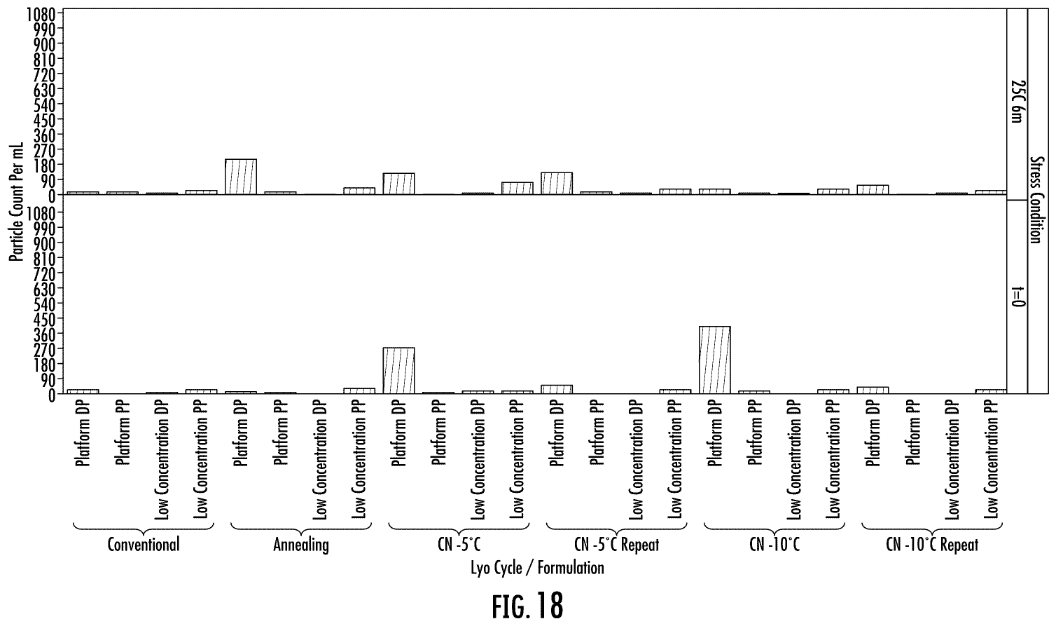

Micro-flow imaging (MFI) of samples from different lyophilization cycles. FIG. 18 depicting MFI analytical data on ≥10 μm unfiltered particles. No substantial impact on subvisible particles was observed.

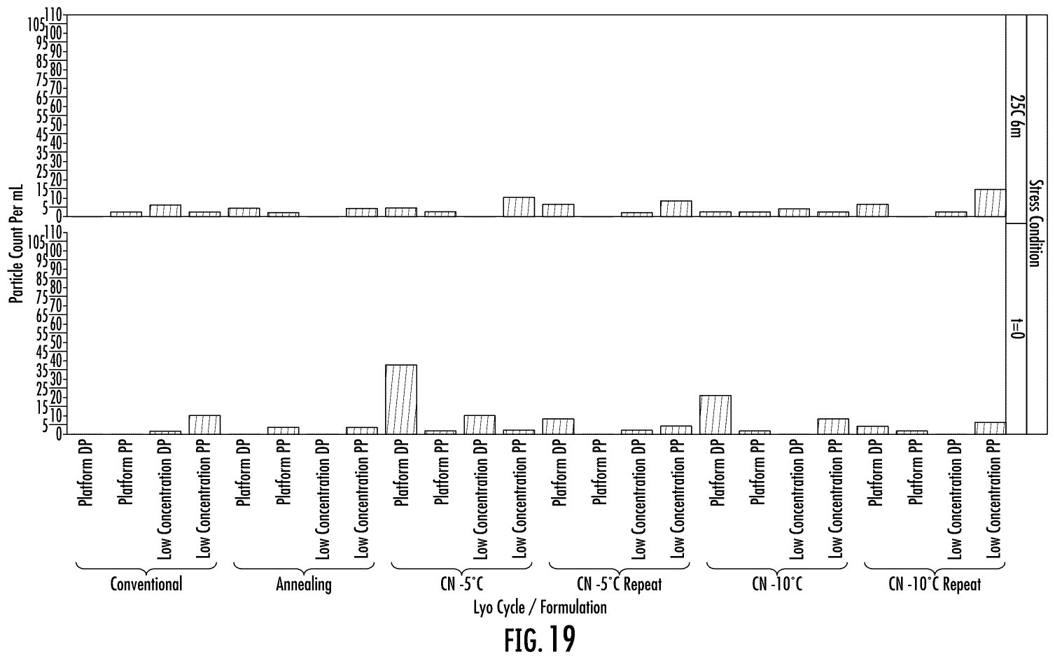

Micro-flow imaging (MFI) of samples from different lyophilization cycles. FIG. 19 depicting MFI analytical data on ≥25 μm unfiltered particles. No substantial impact on subvisible particles was observed.

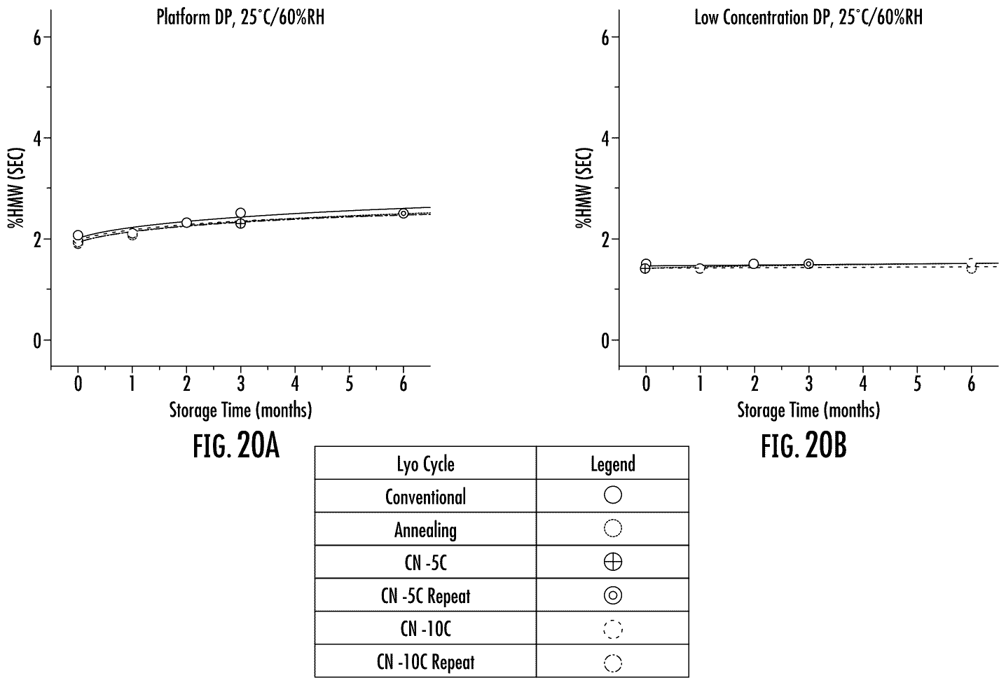

Stability studies were performed on platform DP (FIG. 20A) and low concentration DP (FIG. 20B) samples from different lyophilization cycles for over six months of storage. High molecular weight percent (% HMW) were determined by SEC. Samples were stored at about 25° C. under about 60% relative humidity (RH). FIGS. 20A and 20B showing no substantial impact on stability based on % HMW aggregates as determined by SEC.

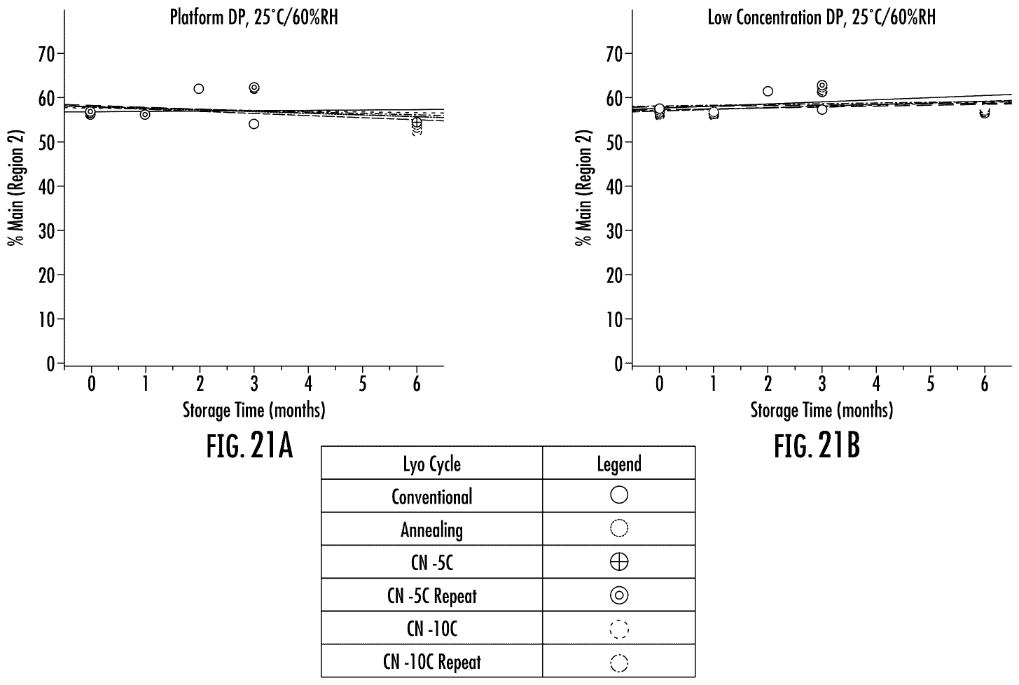

Stability studies were performed on platform DP (FIG. 21A) and low concentration DP (FIG. 21B) samples from different lyophilization cycles for over six months of storage. Samples were stored at 25° C. under 60% relative humidity (RH). FIGS. 21A and 21B showing no substantial impact on stability based on % Main (Region 2) as determined by cation exchange chromatography (CEX).

### Example 13—Protein Concentration and Recovery

Protein concentrations and recovery percent (% Recovery) in formulations F1-F4 (see Table 1) stored for three months at 25° C. and 40° C. were determined using Reverse Phase-Ultra Performance Liquid Chromatography (RP-UPLC). No significant change in protein concentrations was observed (that is, almost full protein recovery observed for all formulations, F1-F4) (Table 6).

Table 6 shows data from RP-UPLC on mAb3 protein concentration and % recovery.

TABLE 6

| Sample Name | Concentration | % Recovery |
|---|---|---|
| 25° C. 3 m F1 | 158 | 98% |
| 25° C. 3 m F2 | 159 | 99% |
| 25° C. 3 m F3 | 162 | 99% |
| 25° C. 3 m F4 | 161 | 97% |
| 40° C. 3 m F1 | 158 | 98% |
| 40° C. 3 m F2 | 158 | 98% |
| 40° C. 3 m F3 | 161 | 99% |
| 40° C. 3 m F4 | 160 | 98% |

### Example 14—Effect of Cryoprotectant on Subvisible Particles

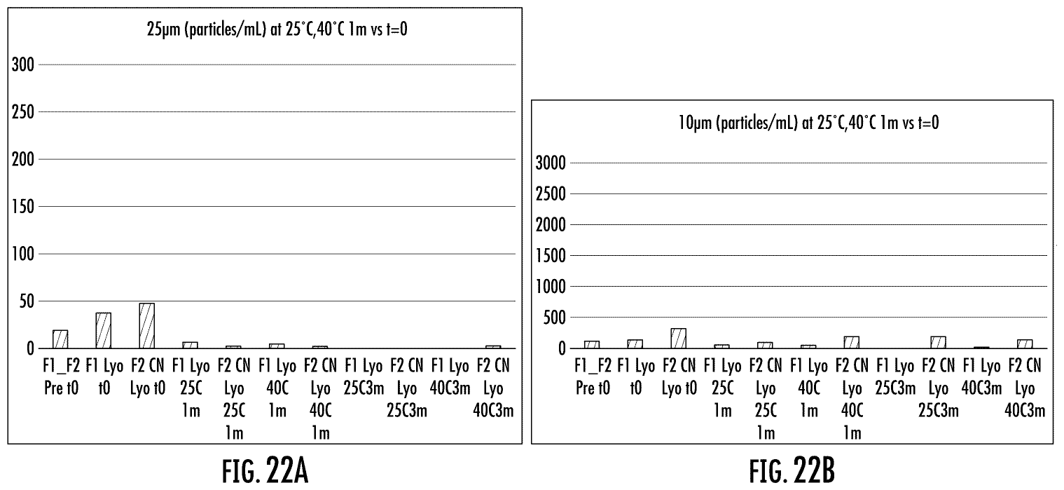

Five percent sucrose containing drug product (DP) formulations F1 and F2 samples from different lyophilization cycles, stored at 25° C. and 40° C. at T₀ and one month were analyzed. FIGS. 22A and 22B are graphs showing data on 25 μm (FIG. 22A) and 10 μm (FIG. 22B) particles in DP formulations, at different temperatures (25° C. and 40° C.) and times (at t₀, 1 m and 3 m). Formulation F1 is Platform Cycle containing 150 mg/ml mAb3, 10 mM L-Histidine, 5% w/v sucrose, 0.1% w/v PS80, at pH 6.0; F2 is CN-5° C. Cycle containing 150 mg/ml mAb3, 10 mM L-Histidine, 5% w/v sucrose, 0.1% w/v PS80, at pH 6.0. No significant impact on particles for 10 μm and 25 μm.

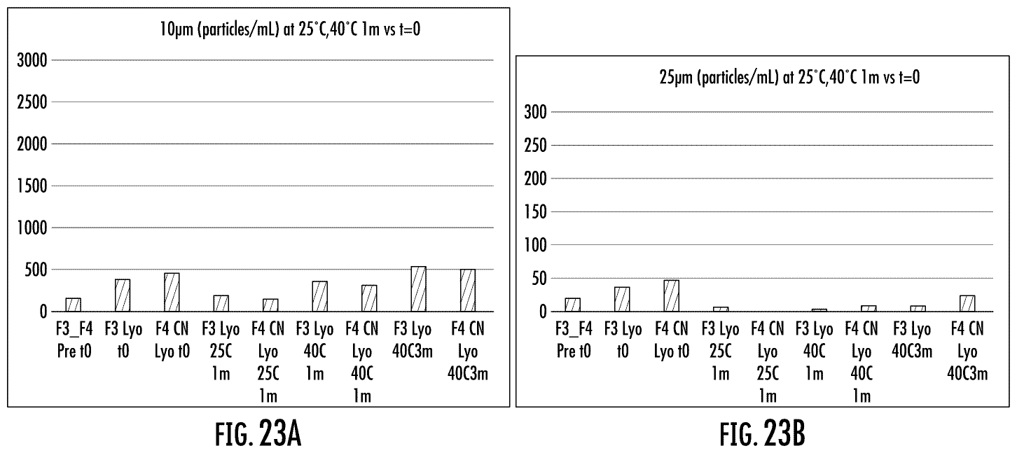

Five percent sucrose containing drug product (DP) formulations F3 and F4 samples from different lyophilization cycles, stored at 25° C. and 40° C. at T₀ and one month were analyzed. FIGS. 23A and 23B are graphs showing data on 10 μm and 25 μm particles, respectively, in DP formulations, at different temperatures (25° C. and 40° C.) and times (at to, 1 m and 3 m). Formulation F3 is Platform Cycle containing 150 mg/ml mAb3, 10 mM L-Histidine, 1.5% w/v sucrose, 0.1% w/v PS80, at pH 6.0; F4 is CN −5° C. Cycle containing 150 mg/ml mAb3, 10 mM L-Histidine, 1.5% w/v sucrose, 0.1% w/v PS80, at pH 6.0. No significant impact on particles for 10 μm and 25 μm.

Results of the experiments disclosed herein reveals that controlled nucleation promotes small and consistent supercooling for ice crystallization (i.e., larger ice crystal formation and thinker cake wall). Large ice crystals impose a lower resistance to water vapor flow from the ice sublimation interface and thinker wall improves cake structures.

Benefits of controlled nucleation include:
Faster Primary Drying and Shorter Cycle,
Reduced reconstitution times,
Improved cake appearance, and
Higher monomer purity post lyophilization.

### Example 15-BET (Brunauer, Emmet, and Teller) Surface Area and Hg Porosimetry Analysis on Lyophilized Cake Samples

Examination of the N₂ BET surface area and Hg porosimetry on lyophilized cake samples were performed. BET theory is a popular model used to determine the surface area of the exposed surface of a solid sample on a molecular scale. Generally, BET surface area analysis is performed using nitrogen (N₂) gas as the adsorbate due to its high affinity for solid surfaces. N₂ gas is introduced at low pressures and the amount adsorbed is determined to calculate the surface area using the BET equation.

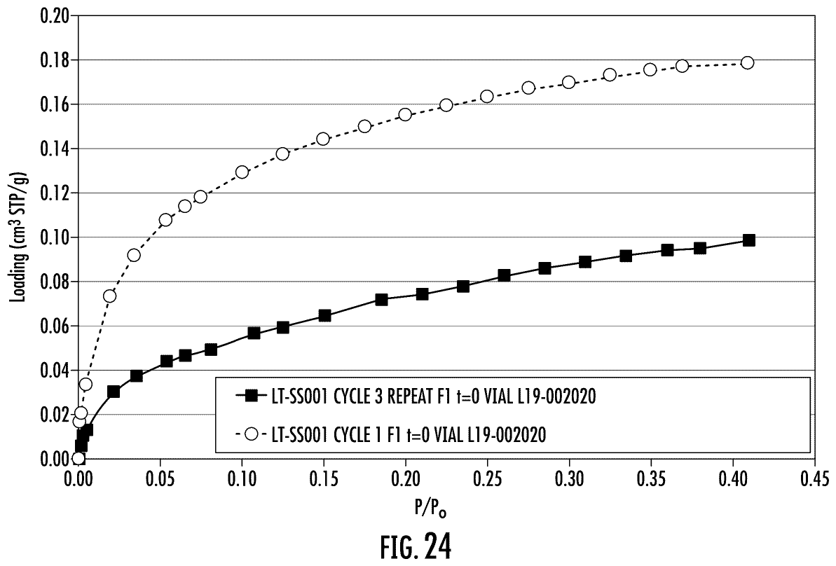

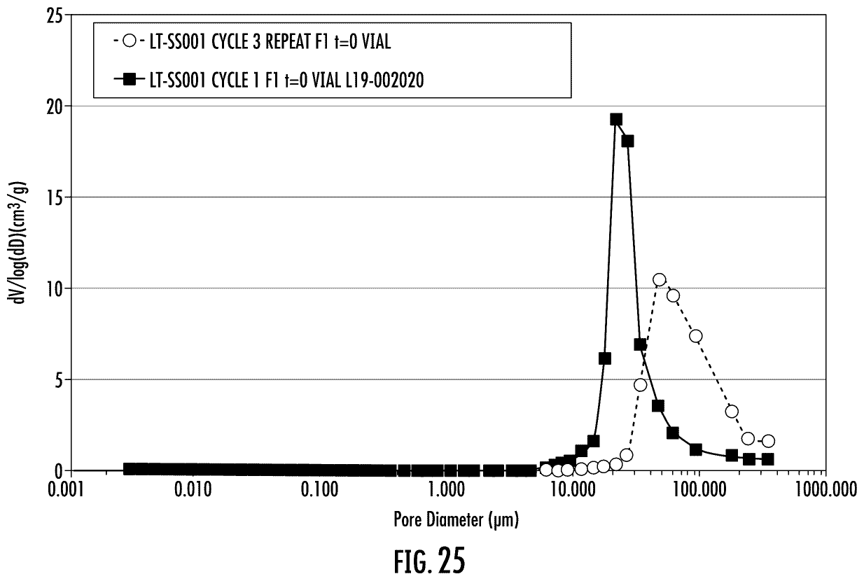

BET analysis show higher specific surface area in conventional vs controlled ice nucleation sample (see Table 7). Additional measured pore diameter via Hg intrusion volume was performed, showing clear difference between conventional vs controlled ice (see FIG. 25), where conventional has much smaller pore diameter (see Table 8). The N₂ isotherm shows that the samples have relatively low surface areas. The isotherm data are provided in FIG. 24. The BET surface area results are summarized in Table 7.

TABLE 7
Summary of the N₂ sorption analysis.

| | Sample | BET Surface |
|---|---|---|
| | Weight | Area |
| Sample | (g) | (m²/g) |
| Lyophilized Drug product from −5° C. | 0.449 | 0.3 |
| Controlled Nucleation Cycle | | |
| (LT-SS001 CYCLE 3 REPEAT F1 t = 0 VIAL | | |
| L19-002020) | | |
| Lyophilized Drug product from Conventional | 0.397 | 0.6 |
| Lyophilization Cycle | | |
| (LT-SS001 CYCLE 1 F1 t = 0 VIAL L19- | | |
| 002020) | | |

TABLE 8
Summary of the Hg porosimetry results.

| | MIV* | D_{Hg} | % Porosity |
|---|---|---|---|
| Sample | (cm³/g) | (g/cm³) | from Hg |
| Lyophilized Drug product | 6.568 | 0.152 | 99.6 |
| from −5° C. Controlled | | | |
| Nucleation Cycle | | | |
| (LT-SS001 CYCLE 3 | | | |
| REPEAT F1 t = 0 VIAL | | | |
| L19-002020) | | | |
| Lyophilized Drug product | 6.375 | 0.140 | 89.5 |
| from Conventional | | | |
| Lyophilization Cycle | | | |
| (LT-SS001 CYCLE 1 F1 | | | |
| t = 0 VIAL L19-002020) | | | |

### N₂ Sorption

The N₂ isotherm was measured on a Micromeritics TriStar II Plus unit to a relative pressure (P/P₀) of approximately 0.4 to enable BET surface area analyses to be performed. The sample was activated on a Smart VacPrep degas unit by degassing at ambient temperature under dynamic vacuum prior to analysis.

Measurements of N₂ BET on standard materials suggest accuracy to within approximately 5% at surface area values of 10 m²/g and approximately 10% at levels of ˜0.5 m²/g when at least 50 m² of material are available for testing within the sample cell. Repeatability for any given sample is dependent on the ability to regenerate the sample to the same degree of activation without modifying the surface or the pore structure.

### Hg Porosimetry

Mercury intrusion experimentation was outsourced. The Hg porosimetry experiments were performed by loading the sample into a chamber of known volume and initially filling the void space around the material with Hg at a filling pressure of ˜0.5 psia. An assumption is made here that no Hg penetrates into the sample at this filling pressure. The Hg pressure was subsequently increased to ˜60,000 psia to force intrusion into the void space within the sample. The amount intruded versus pressure was recorded and converted to a pore size distribution.

The bulk density from Hg porosimetry, often termed the Hg density (pHg), reflects the density of the sample prior to Hg intruding the material and as such is analogous to the geometric density of the material. The units of Hg density, therefore, are grams of solid per volume of solid +void space (assuming no closed porosity). The mercury intrusion volume (MIV), measured by forcing Hg under pressure into the sample, is taken to be void space within the sample. A comparison of the Hg density and the MIV values enables one to estimate the total porosity of the material. The % porosity can be calculated from the following equation:

$$\% Porosity = MIV \times {ρ}_{Hg} \times 100$$

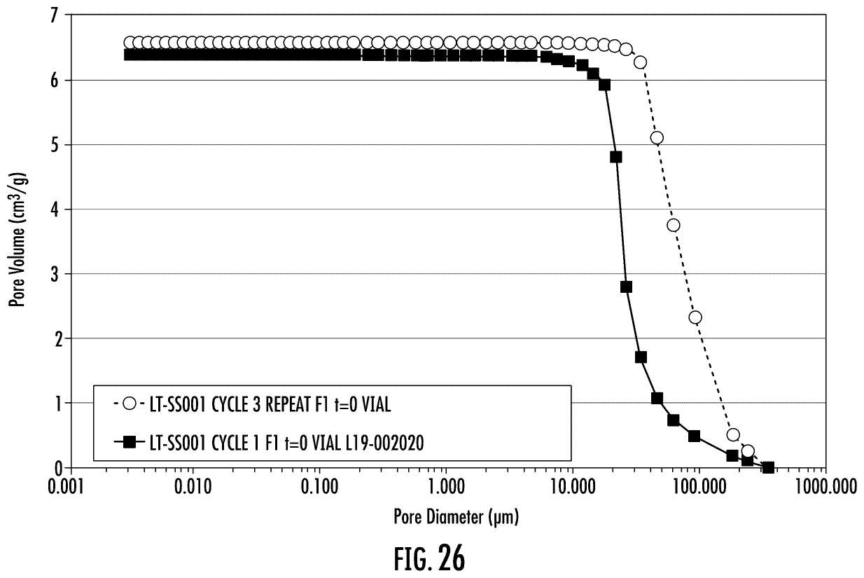

The log differential data for the samples indicate a monomodal PSD. The pore mode for sample LT-SS001 CYCLE 3 REPEAT F1 t=0 VIAL L19-002020 is centered at approximately 45.2 μm, while the pore mode for sample LT-SS001 CYCLE 1 F1 t=0 VIAL L19-002020 is centered at approximately 21.3 μm. The values for the Hg density (i.e., the bulk density at ˜0.5 psia), the MIV, and the calculated porosity are provided in Table 8. The log differential and cumulative volume PSD plots are provided in FIGS. 25 and 26, respectively.

It is to be understood that the description, specific examples and data are given by way of illustration and are not intended to limit the present inventions. Various changes and modifications within the present inventions, including combining teaching in whole and in part, will become apparent to the skilled artisan from the discussion, disclosure and data contained herein, and thus are considered part of the inventions.

1. A method of lyophilizing a protein product, wherein the method comprises the steps:
(a) cooling a solution comprising a protein product below the freezing point of the solution;
(b) pressuring the cooled solution of step (a) with a gas;
(c) releasing the pressure of step (b) to allow nucleation, thereby resulting ice nuclei to form in the cooled solution; and
(d) lyophilizing the cooled solution of step (c) to form a lyophilized protein product.

2. The method of claim 1, wherein the gas is selected from the group consisting of air, helium, nitrogen or argon.

3. The method according to claim 1, wherein during step (b) the gas is present at 14 to 42 psig.

4. The method according to claim 1, wherein during step (b) the gas is present at 25 to 30 psig.

5. The method according to claim 1, wherein the cooled solution is in a lyophilization vial.

6. The method according to claim 1, wherein step (c) is conducted at a temperature of −2° C. to −10° C.

7. The method according to claim 6, wherein step (c) is conducted at a temperature of −5° C.

8. The method according to claim 6, wherein the temperature is maintained for about an hour or less, about 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 minutes or less.

9. (canceled)

10. The method according to claim 1, wherein the protein is an Fc-containing protein.

11. The method according to claim 10, wherein the Fc-containing protein is an Fc-fusion protein.

12. The method according to claim 10, wherein the Fc-containing protein is an antibody.

13. The method according to claim 12, wherein the antibody is a monoclonal antibody.

14. The method according to claim 12, wherein the antibody can be reconstituted to 150 mg/ml in about 3 minutes, about 2 minutes and 50 seconds, about 2 minutes and 40 seconds, about 2 minutes and 30 seconds, about 2 minutes and 20 seconds, about 2 minutes and 10 seconds, about 2 minutes and 5 seconds, or about 1 minute and 45 seconds.

15.-21. (canceled)

22. A lyophilized a protein product produced by a method according to claim 1.

23. A method of making a room temperature stable lyophilized therapeutic protein product by providing a target residual moisture content in the room temperature stable lyophilized therapeutic protein product, wherein the method comprises the steps of:
(a) controlling the shelf temperature (Ts) after ice sublimation without a secondary drying step; and
(b) controlling the duration of post primary drying time;
wherein the target residual moisture content in the lyophilized therapeutic protein product is about 1% to about 7%.

24. The method according to claim 23, wherein the target residual moisture content in the lyophilized therapeutic protein product is about 1.0%, about 1.5%, about 2.0%, 2.5%, about 3.0%, about 3.5%, about 4.0%, about 4.5%, about 5.0%, about 5.5%, about 6.0%, about 6.5%, or about 7.0%.

25. The method according to claim 23, wherein stability of the lyophilized therapeutic protein product is measured by determining change in % HMW protein aggregates in the product.

26. The method according to claim 25, wherein the change in % HMW protein aggregates in the stable lyophilized therapeutic protein product is less than 1.0% as determined by SEC-UPLC.

27. The method according to claim 25, wherein the change in % HMW protein aggregates in the stable lyophilized therapeutic protein product is about 1.0%, about 0.9%, about 0.8%, about 0.7%, about 0.6%, about 0.5%, about 0.4%, about 0.3%, about 0.2%, about 0.1% or about 0%.

28. A room temperature stable lyophilized therapeutic protein product produced by a method according to claim 23.
